SEQUENCE LISTING (10 sequences)

SEQ ID NO: 1 - DNA, 24 bp, synthetic construct
  source 1..24: mol_type = other DNA
  misc_feature 1..24: note = PCR_primers_fwd_seq
acttgcctcctaatactccactca

SEQ ID NO: 2 - DNA, 24 bp, synthetic construct
  source 1..24: mol_type = other DNA
  misc_feature 1..24: note = PCR_primers_rev_seq
atcttcgtctttcatcatttgtcc

SEQ ID NO: 3 - DNA, 20 bp, synthetic construct
  source 1..20: mol_type = other DNA
  misc_feature 1..20: note = PCR_primers_fwd_seq
taagttccagctggctgtgg

SEQ ID NO: 4 - DNA, 20 bp, synthetic construct
  misc_feature 1..20: note = PCR_primers_rev_seq
  source 1..20: mol_type = other DNA
ataggtgggatgggagtgct

SEQ ID NO: 5 - DNA, 24 bp, synthetic construct
  misc_feature 1..24: note = PCR_primers_fwd_seq
  source 1..24: mol_type = other DNA
ctcctatctctacacccacaagcc

SEQ ID NO: 6 - DNA, 23 bp, synthetic construct
  source 1..23: mol_type = other DNA
  misc_feature 1..23: note = PCR_primers_rev_seq
gaatcgccaagtgaatcagagtc

SEQ ID NO: 7 - DNA, 20 bp, synthetic construct
  misc_feature 1..20: note = PCR_primers_fwd_seq
  source 1..20: mol_type = other DNA
ggctggaagctaaaccctgt

SEQ ID NO: 8 - DNA, 20 bp, synthetic construct
  misc_feature 1..20: note = PCR_primers_rev_seq
  source 1..20: mol_type = other DNA
tccgatgctgtaggttgctg

SEQ ID NO: 9 - DNA, 25 bp, synthetic construct
  misc_feature 1..25: note = PCR_primers_fwd_seq
  source 1..25: mol_type = other DNA
ctcatggactgattatggacaggac

SEQ ID NO: 10 - DNA, 25 bp, synthetic construct
  misc_feature 1..25: note = PCR_primers_rev_seq
  source 1..25: mol_type = other DNA
gcaggtcagcaaagaacttatagcc

(19) United States
(12) Patent Application Publication
(10) Pub. No.: US 2024/0374895 A1
(43) Pub. Date: Nov. 14, 2024

(54) TRANSCORNEAL STIMULATOR FOR TREATING BRAIN DISEASES

(71) Applicants: City University of Hong Kong, Hong Kong (HK); The University of Hong Kong, Hong Kong (HK)

(72) Inventors: Lai Hang Leanne CHAN, Hong Kong (HK); Lee Wei LIM, Hong Kong (HK); Wing Shan YU, Hong Kong (HK); Stephen Kugbere AGADAGBA, Hong Kong (HK)

(21) Appl. No.: 18/621,152

(22) Filed: Mar. 29, 2024

Related U.S. Application Data
(60) Provisional application No. 63/500,918, filed on May 9, 2023.

(51) Int. Cl. A61N 1/36; A61N 1/04

(52) U.S. Cl. CPC A61N 1/36025; A61N 1/0408; A61N 1/0472; A61N 1/36031; A61N 1/36034

(57) ABSTRACT

A transcorneal electrical stimulation (TES) apparatus for treating brain diseases is provided. The apparatus includes an eye-contacting interface with at least one active electrode, an inactive reference electrode, and a current source. Both the active and reference electrodes are electrically connected to the current source, facilitating transcorneal electrical stimulation for various applications.

### CROSS-REFERENCE TO RELATED APPLICATIONS

The present application claims priority from U.S. provisional patent application Ser. No. 63/500,918 filed May 9, 2023, and the disclosure of which is incorporated herein by reference in its entirety.

### REFERENCE TO SEQUENCE DISCLOSURE

The sequence listing file under the file name “P2740US01_Sequence Listing.xml” submitted in ST.26 XML file format with a file size of 13.4 KB created on Mar. 11, 2024 and filed on Mar. 29, 2024 is incorporated herein by reference.

### FIELD OF THE INVENTION

The present invention generally relates to the field of neurosciences. More specifically the present invention relates to eye stimulation therapy apparatus and methods of treating brain diseases through the eye-brain connection.

### BACKGROUND OF THE INVENTION

The brain is one of the most complex and important organs in the body, responsible for maintaining homeostasis and coordinating bodily functions. Brain diseases encompass a wide range of conditions affecting the brain's structure and function, often leading to cognitive, emotional, and behavioral disturbances.

Depressive disorder is often considered a brain disease. It is a mental health condition that affects a person's mood, thoughts, and overall functioning. While it involves psychological and emotional aspects, research has shown that there are also underlying neurological and neurochemical factors associated with depressive disorders. Additionally, structural and functional changes in certain areas of the brain have been identified in people experiencing depression. It is characterized by recurrent sadness, low motivation, negative thinking and hopelessness which may culminate in suicide, and now depression is a significant contributor to the global disability burden.

Furthermore, dementia, which is not a specific disease but a term encompassing a group of symptoms associated with a decline in cognitive function that interferes with daily life, leads to pathological deterioration in cognitive and memory functions. While dementia is not considered a normal part of aging, its incidence increases with age, from 5% in those aged 65-74 to 35% in those aged 85 or above. Globally, dementia is the seventh leading cause of death among all diseases. Dementia is an immense global burden, with an estimation of 50 million people worldwide affected and an annual cost of one trillion US dollars. Despite much research on its various types and manifestations, there is still no effective prevention or cure for dementia because of its multifactorial and complex etiology, adding to the burden of dementia. Alzheimer's disease is the most common accounting for 60-80% of all cases and most harmful form of dementia characterized by the presence of amyloid beta (Aβ) plaques in the brain.

Currently, there is no effective cure for both depressive disorder and dementia. However, there is a close association between the visual and emotional systems as reflected by the extensive interconnections of the cortical visual inputs to the subcortical limbic regions. The visual and emotional systems are extensively interconnected at the cortical and subcortical levels, with visual inputs transmitted to the limbic regions, including the hippocampus and amygdala.

Further, an association between dementia and visual impairment has been widely documented. Previous studies observed a higher risk of dementia among individuals with low vision. The eyes (specifically the retinas) are considered to be extensions of the brain because of multiple shared features, including a common embryological origin of the forebrain and eyes from the anterior neural plate, comparable morphology of the brain and retinal neurons, structural and functional parallels in the vasculature and blood-tissue barriers, the presence of similar types of neurotransmitters, and the presence of immunoregulatory mediators in the aqueous humor that is reminiscent of the cerebrospinal fluid.

Transcorneal electrical stimulation (TES) is a non-invasive technique conventionally used for the potential restoration of vision in blind patients. The stimulation parameters can be easily fine-turned depending on the disease progression. However, the focus of TES research has been on improving visual functions, while its effects on neuropsychiatric disorders remains unexplored. Most of the studies on TES have focused on improving visual functions, while its effects on non-visual systems, especially its psychiatric effects, have not been extensively explored.

Therefore, the present invention addresses the need for providing a safe but effective apparatus for non-pharmaceutical treatment of brain diseases with no side effects, such as weight change, hepatotoxicity, gastrointestinal problems, dizziness, headache, confusion and agitation.

### SUMMARY OF THE INVENTION

It is an objective of the present invention to provide methods and apparatus to solve the aforementioned technical problems.

In accordance with a first aspect of the present invention, a transcorneal electrical stimulation (TES) apparatus for treating a brain disease is provided. Specifically, the apparatus includes an eye contacting interface with at least one active electrode, an inactive reference electrode, and a current source. The active electrode and the inactive reference electrode are connected to the current source.

In accordance with one embodiment of the present invention, the apparatus further includes a control unit configured to receive data related to the patient's brain activity and adjust the current parameters in real-time based on the received data.

In accordance with one embodiment of the present invention, the control unit is connected to the current source

In accordance with one embodiment of the present invention, the current provider is programmable, allowing healthcare providers to customize treatment protocols for individual patients.

In accordance with one embodiment of the present invention, the brain disease comprises a depressive disorder and a dementia.

In accordance with one embodiment of the present invention, the current provider is an electrical pulse generator for controlling the delivery of electric current to the eye.

In accordance with one embodiment of the present invention, the eye contacting interface includes a contact lens.

In accordance with one embodiment of the present invention, the contact lens is configured with a material that enhances electrode-skin contact and patient comfort.

In accordance with a second aspect of the present invention, a method of treating a brain disease in a patient in need utilizing the aforementioned TES apparatus is introduced. Particularly, the method includes placing the eye contacting interface upon a patient's eye and the inactive reference electrode on a skin area of the patient; and providing a current through the electrodes for a duration.

In accordance with one embodiment of the present invention, wherein the current is a 100-200 μA current.

In accordance with one embodiment of the present invention, the brain disease includes a depressive disorder and a dementia.

In accordance with one embodiment of the present invention, the method further includes a step of adjusting the duration and intensity of the current based on the patient's need and capacity.

In accordance with another embodiment of the present invention, the method further includes a step of monitoring the patient's physiological responses during the treatment and adjusting the current accordingly.

### BRIEF DESCRIPTION OF THE DRAWINGS

Embodiments of the invention are described in more details hereinafter with reference to the drawings, in which:

FIG. 1 depicts a TES apparatus of one embodiment of the present invention;

FIGS. 2A-2B depict the TES procedures in different animal models, wherein FIG. 2A displays a schematic representation of the timeline and procedure of TES in S334ter-line-3 rats and FIG. 2B depicts a schematic representation of the timeline and set-up for TES in the CUS model;

FIG. 3 depicts a schematic diagram representing the in vivo recording of retinal-evoked activity in the superior colliculus;

FIGS. 4A-4D show the behavioral outcomes in S334ter-line-3 rats, FIG. 4A exhibits the results of cylinder test; FIG. 4B depicts the results of OFT; FIG. 4C depicts the results of HCET; and FIG. 4D shows the results of FST;

FIG. 5 exhibits the response latencies of neurons in the superior colliculus;

FIG. 6 shows EEPs in both S334ter-line-3 and control rats;

FIGS. 7A-7C show H&E staining of normal and degenerated retinas; FIG. 7A is a set of representative images of H&E staining of the retina of control and S334ter-line-3 rats with sham or TES treatments; FIG. 7B indicates the comparison of the thickness of the inner nuclear layer in control and S334ter-line-3 rats with sham or TES treatments; and FIG. 7C shows the comparison of the thickness of the inner plexiform layer in control and S334ter-line-3 rats with sham or TES treatments;

FIGS. 8A-8F show the effects of TES on behavioral outcomes, plasma corticosterone level, and hippocampal neurogenesis-related gene expression in the CUS rat model; FIG. 8A depicts the results of OFT; FIG. 8B shows the outcomes of SuPT; FIG. 8C exhibits the results of FST; FIG. 8D shows the plasma corticosterone level in the CUS model; FIG. 8E demonstrates real-time PCR analysis of neurogenesis-related genes in hippocampus; and FIG. 8F is a scatter plots diagram displaying correlations between Ki67 and NeuN;

FIGS. 9A-9F depict blot images and densitometric analysis thereof showing protein expression related to neuroplasticity and cell death in hippocampus of CUS model; FIG. 9A shows the blot images of hippocampus of CUS model; FIG. 9B shows pAKT/AKT ratio; FIG. 9C depicts pPKA/PKA ratio; FIG. 9D depicts the expression of SYP; FIG. 9E exhibits the expression of PSD95; and FIG. 9F depicts the expression of BAX;

FIGS. 10A-10F depict blot images and densitometric analysis thereof showing protein expression related to neuroplasticity and cell death in amygdala of CUS model; FIG. 10A shows the blot images of amygdala of CUS model; FIG. 10B shows pAKT/AKT ratio; FIG. 10C depicts pPKA/PKA ratio; FIG. 10D depicts the expression of SYP; FIG. 10E exhibits the expression of PSD95; and FIG. 10F depicts the expression of BAX;

FIGS. 11A-11C exhibit schematic representations of timeline; FIG. 11A shows the example design of aged mice model proving that aged mice having memory deficits in spatial memory and locomotor activity; FIG. 11B depicts the example design of 5XFAD mice model showing improvement in Y-maze spatial memory and MWM spatial learning performance; FIG. 11C depicts another example design of 5XFAD mice showing improvement in spatial learning performance;

FIGS. 12A-12D show that the aged mice model having memory deficits in spatial memory ad locomotor activity; FIG. 12A exhibits the Y-maze test results of young and aged mice; FIG. 12B shows that aged and young mice differ in the distance moved in the OFT; FIG. 12C depicts the MWM test outcomes of young and aged mice; and FIG. 12D shows the result diagram of the MWM probe test;

FIGS. 13A-13E show that TES improves spatial learning and memory in aged mice; FIG. 13A depicts the apparatus for TES; FIG. 13B depicts the outcomes of Y-maze test; FIG. 13C shows the results of OFT test; FIG. 13D shows the observations of MWM training phase; and FIG. 13E depicts the results of MWM probe test;

FIGS. 14A-14E show that TES improves Y-maze spatial memory and MWM spatial learning performance in male 5XFAD mice; FIG. 14A depicts the outcomes of Y-maze test; FIG. 14B shows the results of OFT test; FIG. 14C shows the observations of MWM training phase; FIG. 14D depicts the results of MWM probe test; and FIG. 14E depicts representative swimming tracks during the MWM probe test;

FIGS. 15A-15D prove that TES ameliorate β-amyloid deposition in the hippocampus of male 5XFAD mice; FIG. 15A depicts representative images of β-amyloid staining in the dorsal hippocampus of male 5XFAD mice; FIG. 15B depicts representative images of β-amyloid staining in the ventral hippocampus of 5XFAD mice; FIG. 15C shows amyloid burden in the dorsal hippocampal subregions; FIG. 15D exhibits analysis of amyloid burden;

FIGS. 16A-16E show that TES upregulates the expression of PSD95 in the hippocampus of male 5XFAD mice; FIG. 16A shows western blot images of associated markers in the dorsal and ventral hippocampi; FIG. 16B is a densitometric analysis of SYP in the dorsal and ventral hippocampi; FIG. 16C depicts a densitometric analysis of PSD95 protein expression in the dorsal and ventral hippocampi; FIG. 16D shows a densitometric analysis of IBA-1 in the dorsal and ventral hippocampi; and FIG. 16E shows a densitometric analysis of GFAP in the dorsal and ventral hippocampi;

FIGS. 17A-17E show that TES improves spatial learning performance in female 5XFAD mice; FIG. 17A depicts the Y-maze test results; FIG. 17B shows the outcomes of OFT; FIG. 17C exhibits the learning performance during the MWM training phase; FIG. 17D shows the MWM probe test results; and FIG. 17E depicts representative swimming tracks during the MWM probe test; and

FIGS. 18A-18D indicate that TES does not affect-amyloid deposition in the hippocampus of female 5XFAD mice; FIG. 18A shows representative images of β-amyloid staining in the dorsal hippocampus of female 5XFAD mice; FIG. 18B exhibits representative images of β-amyloid staining in the ventral hippocampus of female 5XFAD mice; FIG. 18C shows the analysis of amyloid burden; and FIG. 18D demonstrates the amyloid burden in the ventral hippocampal subregions.

### DETAILED DESCRIPTION

In the following description methods and apparatus of treating brain diseases and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

In scientific and medical contexts, the term “brain disease” is often used to describe conditions that primarily affect the structure or function of the brain, leading to impairments in cognition, behavior, or both.

The term “depressive disorders” used herein is commonly known as depression, are mental health conditions characterized by persistent feelings of sadness, hopelessness, and a lack of interest or pleasure in activities. Depression involves complex interactions among neurotransmitters (e.g., serotonin, norepinephrine), brain circuits, and structural changes. Imbalances in these neurotransmitters and alterations in neural pathways contribute to the development and maintenance of depressive symptoms. Structural changes in areas like the hippocampus (involved in memory and emotions) have been observed in individuals with chronic depression. Chronic stress associated with depression may also impact the brain's architecture.

The term “dementia” used herein is characterized by a decline in cognitive function, affecting memory, reasoning, and the ability to perform everyday activities. It is not a specific disease itself but rather a syndrome resulting from various disorders that affect the brain. Alzheimer's disease is the most common cause of dementia, but other conditions, such as vascular dementia, Lewy body dementia, and frontotemporal dementia, also contribute. Since dementia involves structural and functional changes in the brain, including those crucial for memory, language, and decision-making, calling it a brain disease is accurate and appropriate. This terminology helps convey that the roots of the symptoms are in the brain's biological and neurological processes.

Both depressive disorders and dementia involve changes in brain structure and function, and neurotransmitter imbalances and alterations in neural pathways play a role in both conditions.

The term “transcorneal electrical stimulation (TES)” used herein is a non-invasive method of activating the retina and retinofugal pathways. In practice, an active electrode is placed on the corneal surface of the eye and an inactive reference electrode is carefully positioned underneath the skin in close proximity to the eye. The active electrode is connected to an electrical pulse generator that controls the delivery of electric current to the eye. The stimulation parameters such as current amplitude, stimulation frequency, pulse duration, and repetition periods can be adjusted and varied based on each subject and pathological condition.

In accordance with a first aspect of the present invention, an apparatus of treating brain diseases through TES is provided. The apparatus includes an eye-contacting interface equipped with at least one active electrode, an inactive reference electrode, and a current provider. The active and reference electrodes are seamlessly connected to the current provider, ensuring a cohesive and integrated system. Specifically, the active electrode is strategically placed in contact with the patient's eye, while the reference electrode is positioned on a skin area, thereby establishing a reliable electrical circuit for the transcorneal stimulation.

In one embodiment, the apparatus 10 is provided. The eye-contacting interface 101 is in a contact lens form and equipped with at least one active electrode 101a. The at least one active electrode 101a and the inactive reference electrode 102 are connected to the current provider 103.

In some embodiments, the contact lens is configured with a material that enhances electrode-skin contact and patient comfort.

In one embodiment of the invention, the apparatus is designed to incorporate a sophisticated control unit. This control unit is configured to receive real-time data related to the patient's brain activity. The acquired data is then utilized to dynamically adjust the parameters of the current, optimizing the therapeutic effects based on the specific needs and responses of the patient. This real-time feedback mechanism enhances the precision and efficacy of the treatment, aligning with the principles of personalized medicine.

The current provider within the apparatus is programmable, allowing healthcare providers to customize treatment protocols for individual patients. This programmability feature empowers healthcare professionals to tailor the treatment based on the unique characteristics and requirements of each patient, thereby optimizing the therapeutic outcomes.

In some embodiments, the apparatus is capable of treating brain diseases such as depressive disorders and dementia. The versatility of the apparatus extends its application to a range of neurological conditions, thereby broadening its impact in the field of medical treatments.

Crucially, the current provider in the apparatus may be an electrical pulse generator. This generator serves as a pivotal component for controlling the delivery of electric current to the eye. The generator ensures a precise and regulated administration of the transcorneal electrical stimulation, underlining the reliability of the present apparatus in the treatment of various brain diseases.

In accordance with a second aspect of the present invention, a method of treating brain diseases using transcorneal electrical stimulation (TES), a non-invasive, safe, reversible, and highly adjustable visual stimulation approach, is provided.

In this method, an effective and non-invasive approach is employed by placing at least one active electrode within a specialized eye contacting interface onto the patient's eye, while an inactive reference electrode is strategically positioned on a designated skin area. The eye contacting interface, in a preferred embodiment, adopts the form of a contact lens, ensuring direct and efficient electrical contact. The application of a controlled current, ranging between 100-200 μA, through these electrodes over a specified duration constitutes a fundamental aspect of this inventive treatment.

In specific instances, the brain diseases targeted encompass a spectrum, including but not limited to depressive disorders and dementia. The versatility of this method extends to addressing various conditions, enhancing its applicability in the field of neurological disorders. Notably, the method accommodates individual patient needs and capacities by incorporating mechanisms to adjust both the duration and intensity of the administered current.

Moreover, an integral feature of this inventive method involves continuous monitoring of the patient's physiological responses throughout the treatment. This real-time feedback is utilized to dynamically adjust the current, ensuring optimal therapeutic effects while prioritizing patient safety and comfort. The inventive approach is further enhanced by the configuration of the contact lens form, which incorporates materials specifically chosen to augment electrode-skin contact and promote patient comfort.

To facilitate the precise and controlled delivery of electric current, the at least one active electrode is connected to an electrical pulse generator. This generator serves as a sophisticated control unit, overseeing and regulating the parameters of the electrical stimulation. This integration ensures a tailored and adaptive treatment approach, attesting to the innovation and efficacy of the disclosed method in the treatment of various brain diseases.

In the following examples, it is evidenced that TES activates brain pathways, resulting in significant antidepressant effects and reduced stress hormones in rodent models of stress and depression.

Moreover, in the hippocampus, it is shown that TES can induce the expression of genes that facilitate growth and development of brain cells. Surprisingly, it is also proved that TES reduces Aβ plaques and significantly improves memory performance in the hippocampus of a rodent model of Alzheimer's disease.

Presenting by this invention, the embodiments clearly demonstrate the antidepressant effects of TES in rat models of stress and depression (CUS rats and S334ter-line-3 rats). Furthermore, it is also provided that the effects of TES on memory enhancement using two mice models (aged mice and the 5XFAD model of Alzheimer's disease).

### EXAMPLES

### Animal Model

All rats and mice are housed in a temperature- and humidity-controlled room (25±1° C. and 60%-65% humidity) under a 12 h light/dark cycle (lights on at 21:00) with access to food and water ad libitum.

### Rat Model of Retinal Degeneration

Ten-week-old male S334ter-line-3 rats and Long Evans (LE) rats are used as the retinal degeneration model and healthy controls, respectively. The rats are obtained from the Laboratory Animal Services Centre of the Chinese University of Hong Kong. S334ter-line-3 rats are bred from transgenic homozygotes and pigmented LE rats. Details of the generation of S334ter-line-3 rats have been described previously.

### Rat Model of Chronic Unpredictable Stress (CUS)

Twelve-week-old male Sprague-Dawley (SD) rats are obtained from the Laboratory Animal Unit of the University of Hong Kong. The CUS paradigm is performed as previously described. Briefly, SD rats are exposed to various stressors for 3 consecutive weeks. Stressors in the CUS protocol include intermittent illumination every 2 h, stroboscopic illumination (2.5 Hz), water and/or food deprivation, individual housing, crowded housing, damp bedding (300 mL cold tap water), dirty cage with excreta from other rats, and loud noises. One stressor is applied every 12 h daily, with the order of stressors randomized to maintain unpredictability.

### Mice Model of Alzheimer's Disease

Male C57BL/6J mice 18 and 6 months old are used as aged mice and corresponding young controls, respectively. Both male and female 5XFAD mice (6 months old) are used as the model of AD. The 5XFAD mice are purchased from the Jackson Laboratory (Bar Harbor, ME, USA) and maintained by breeding hemizygous transgenic mice with C57BL/6J mice. The wild-type littermates are used as controls. The generation of 5XFAD mice has been described previously; the mice express a human amyloid precursor protein (APP) transgene harboring the Swedish, Florida, and London mutations coupled with a human presenilin 1 (PS1) transgene with M146L and L286V mutations. Overexpression of both transgenes is driven by the neuron-specific mouse Thy1 promoter.

### Example 1. Evaluation of Antidepressant Effects of TES

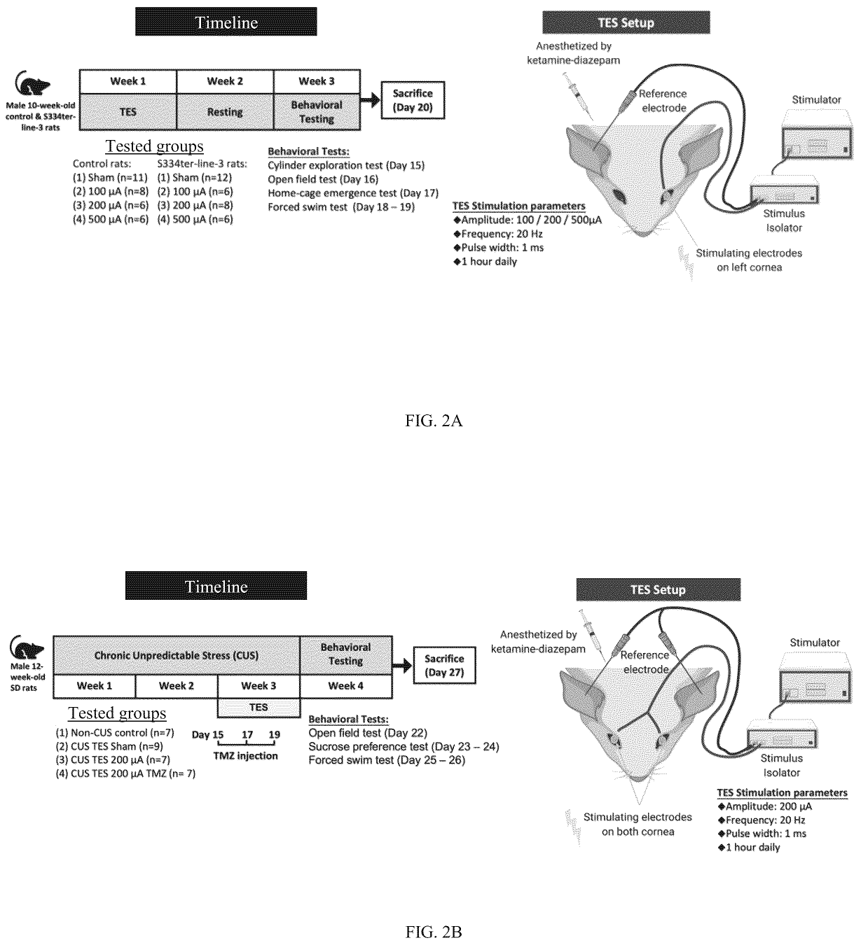

Two systematic studies are conducted to evaluate the antidepressant-like effects of TES. In Study 1, the effects of TES on behavioral outcomes are examined in a rat model of retinal degeneration (FIG. 2A). S334ter-line-3 rats (TES 100 μA, n=6; TES 200 μA, n=8; TES 500 μA, n=6; and sham, n=12) and LE control rats (TES 100 μA, n=8; TES 200 μA, n=6; TES 500 μA, n=6; and sham, n=11) received daily TES or sham treatment for 1 week before behavioral assessments. Anxiety-like behaviors are assessed by cylinder exploration, open field, and home cage emergence tests, whereas behavioral despair is assessed by the forced swim test. Animals are sacrificed one day after the last behavioral assessment and retinal tissues are collected for histological workup. The S334ter-line-3 model has been reported to exhibit progressive loss of visually evoked activity in the superior colliculus which might prevent the transmission of TES-induced input to the visually connected brain regions. To this end, electrophysiology is also conducted in a separate batch of S334ter-line-3 rats to assess the integrity of the visual pathway.

In Study 2, the antidepressant-like effects of TES are examined in a CUS rat model (FIG. 2B). Daily TES or sham treatments are applied in week 3 during the CUS process, whereas the non-CUS control rats are left undisturbed (non-CUS, n=7; TES 200 μA, n=7; sham, n=9). Additionally, a group of TES-treated CUS rats are intraperitoneally injected with TMZ (25 mg/kg, SelleckChem, Texas, USA) on stimulation days 1, 3, and 5 to block cell proliferation so as to assess the neurogenesis-dependent and -independent effects of TES (n=7). Locomotor ability is assessed in the open field test, whereas anhedonia and behavioral despair are measured using the sucrose preference test and forced swim test, respectively [40]. Rats are sacrificed one day after the last behavioral assessment. Blood plasma is collected for measuring corticosterone level and brain tissue is harvested for measuring the expression of hippocampal neurogenesis-related genes. Immunoblotting is carried out to analyze neuroplasticity- and apoptosis-associated markers in the hippocampus and amygdala.

TES is performed by the following procedures. Animals are anesthetized by intraperitoneal injection of ketamine (70 mg/kg) and diazepam (7 mg/kg). A ring-shaped non-invasive stimulating electrode is placed on the cornea and eye moisturizing gel (Dechra, Northwich, UK) is applied to prevent dehydration. Electrical currents are delivered using a digital stimulator model 3800 and stimulus isolators model 3820 (A-M Systems Inc., Sequim, USA) at a frequency of 20 Hz, pulse width of 1 ms, and amplitude of 100, 200, or 500 mA. The parameters are chosen based on previous research that reported TES had neuro-protective effects in the retina. The impedance of the stimulating electrodes was 112±4.2 U at 1 KHz, and 85.1±1.8 U at 100 kHz. The sham groups are similarly treated but without electrical stimulation.

For behavioral testing, all tests are conducted during the dark cycle in dim light condition by researchers blinded to the treatment conditions. Animal behaviors are recorded and scored using the ANY-maze behavioral tracking software (Stoelting Co., USA). For cylinder test, animals are placed in an open-top transparent Plexiglas cylinder (height 50 cm×diameter 20 cm). Immobility and rearing behaviors (vertical exploration while standing on hind legs) are recorded during the 10-min test period.

The open field test (OFT) is conducted in a lid-free white Plexiglas square box (100×100×40 cm³) with a dark floor. Rats are placed in the central area and allowed to explore for 5 min. Behaviors including distance traveled and time spent in the center and peripheral zones are recorded and scored. Home cage emergence test (HCET), briefly, the animal's home cage is placed on a platform with the cage lid removed. A grid walkway is placed over the edge of the home cage to facilitate escape. The escape latency, which is the time to emerge from the home cage, is recorded and scored. If rats do not escape the home cage within 10 min, they are given a score of 600 s.

In sucrose preference test (SuPT), rats are habituated to drinking 1% sucrose solution for 1 h, and then restricted to food and water for 14 h. Thereafter, rats are provided bottles of pre-weighed water and 1% sucrose solution for 1 h. Sucrose preference is calculated as the percentage of sucrose consumed relative to the total liquid intake. In order to perform forced swim test (FST), rats are placed in a cylindrical Plexiglas tank (height 50 cm×diameter 20 cm) filled with tap water (25±1° C.) to a height of 30 cm. Rats are allowed to swim in the water tank for 15 min in a pretest session to acclimate them to the novel swimming behavior. This is followed by a 10-min test session on the next day. The immobility duration, defined as a period of no movement or slight and infrequent movements with the nose maintained above water, is recorded.

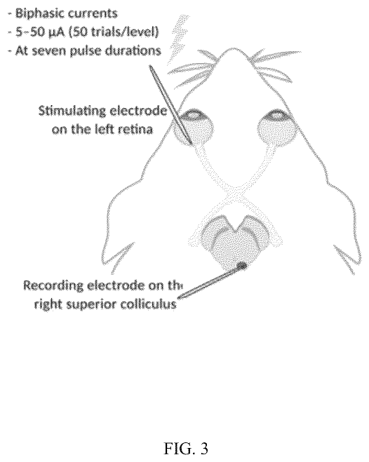

Electrophysiology (FIG. 3), briefly, epiretinal stimulation of the left eye is conducted in S334ter-line-3 (n=5) and control (n=4) rats. Charge-balanced biphasic currents (5-50 μA, 50 trials per threshold level) are applied to the retinal surface across three pulse durations (0.5, 1.0, and 1.5 ms). Retinal evoked responses from the contralateral superior colliculus are recorded.

The histology of retinal is also evaluated. Briefly, rats are euthanized one day after the last behavioral assessment using sodium pentobarbital. The stimulated eyes of S334ter-line-3 and LE rats are enucleated and fixed in 4% paraformaldehyde in phosphate buffered solution at 4° C. overnight. Samples are cryoprotected in 15% and 30% sucrose solution overnight and subsequently frozen in liquid nitrogen. Samples are sectioned into 20-mm thick slices using a cryostat. Standard hematoxylin-cosin (H&E) staining is performed. Images of the stained retina are obtained at 20× using a Zeiss Axioplan 2 microscope equipped with an Olympus DP73 camera. Thickness of the retinal layers in the central region (1 mm from the optic disk) is measured with image processing software.

One day after the last behavioral assessment, animals are sacrificed. Blood is collected in sampling tubes containing EDTA (1.5 mg/mL) and centrifuged at 14000 rpm for 5 min at 4° C. The supernatant is collected as plasma and extracted with dichloromethane, followed by vortexing for 1 min. Corticosterone level is determined directly from the dried dichloromethane extracts by radioimmunoassay using corticosterone-I. The radioimmunological reaction is conducted overnight at 4° C., followed by the separation of unbound steroids.

The hippocampus is grossly dissected from freshly frozen brain tissues of the CUS rats. Total RNA is extracted from the hippocampus using a ready-to-use reagent for isolation of the isolation of high-quality total RNA or the simultaneous isolation of RNA, DNA, and protein from a variety of biological samples and reverse transcribed using cDNA generate kit with gDNA eraser. Real-time PCR is performed and hypoxanthine guanine phosphoribosyltransferase (Hprt) is used as the internal control for mRNA expression. Fold change is determined relative to the TES sham group after normalizing to Hprt using the 2-(DDCt) method. The primers used herein are as follows:

Ki67:
forward (SEQ ID NO. 01):
5′-ACTTGCCTCCTAATACTCCACTCA-3′;
reverse (SEQ ID NO. 02):
5′-ATCTTCGTCTTTCATCATTTGTCC-3′,
Nestin:
forward (SEQ ID NO. 03):
5′-TAAGTTCCAGCTGGCTGTGG-3′;
reverse (SEQ ID NO. 04):
5′-ATAGGTGGGATGGGAGTGCT-3′,
Dcx:
forward (SEQ ID NO. 05):
5′-CTCCTATCTCTACACCCACAAGCC-3′;
reverse (SEQ ID NO. 06):
5′-GAATCGCCAAGTGAATCAGAGTC-3′)
Neun:
forward (SEQ ID NO. 07):
5′-GGCTGGAAGCTAAACCCTGT-3′;
reverse (SEQ ID NO. 08):
5′-TCCGATGCTGTAGGTTGCTG-3′,
and
Hprt:
forward (SEQ ID NO. 09):
5′-CTCATGGACTGATTATGGACAGGAC-3′;
reverse (SEQ ID NO. 10):
5′-GCAGGTCAGCAAAGAACTTATAGCC-3′.

The amygdala and hippocampus of the CUS rats are rapidly isolated by gross dissection. Total proteins are extracted by homogenization in lysis buffer with a protease and phosphatase inhibitor cocktail, and protein concentration is determined by Bradford assay. Protein samples (10-20 μg) are resolved by 10% SDS-PAGE and then transferred onto PVDF membranes using a semi-dry electroblotting system. The membranes are blocked in 5% milk or BSA in TBS for 1 h at room temperature and incubated at 4° C. overnight with primary antibodies. Primary antibodies against pPKA CThr197 (1:1000), PKA C-a (1:1000), pAKTS473 (1:1000), AKT (1:1000), BAX (1:1000), SYP (1:2000), PSD95 (1:1000), and GAPDH (1:1000) are used. After washing in TBS-T, the membranes are incubated in horseradish peroxidase-conjugated secondary antibody (1:2000) for 2 h at room temperature. Immunoreactive bands are visualized by a western blot imaging system. Densitometric analysis of the bands is performed using Image Lab Software. The relative protein expression is normalized against GAPDH

In study 1, behavioral and electrophysiology data are analyzed using a two-way analysis of variance (ANOVA) followed by Bonferroni post-hoc test for multiple comparisons. Data from the histological study are analyzed by one-way ANOVA with Bonferroni post-hoc test. In Study 2, the results are analyzed by one-way ANOVA followed by multiple comparisons with Bonferroni correction. Spearman correlation coefficients are calculated to examine the association between neurogenesis-related markers and behavioral parameters. All data are presented as mean+S.E.M. Significance is defined as p<0.05.

Behavioral comparisons are performed between S334ter-line-3 and LE control rats given TES or sham treatments to assess the effects of retinal degeneration and TES on anxiety- and depressive-like behaviors.

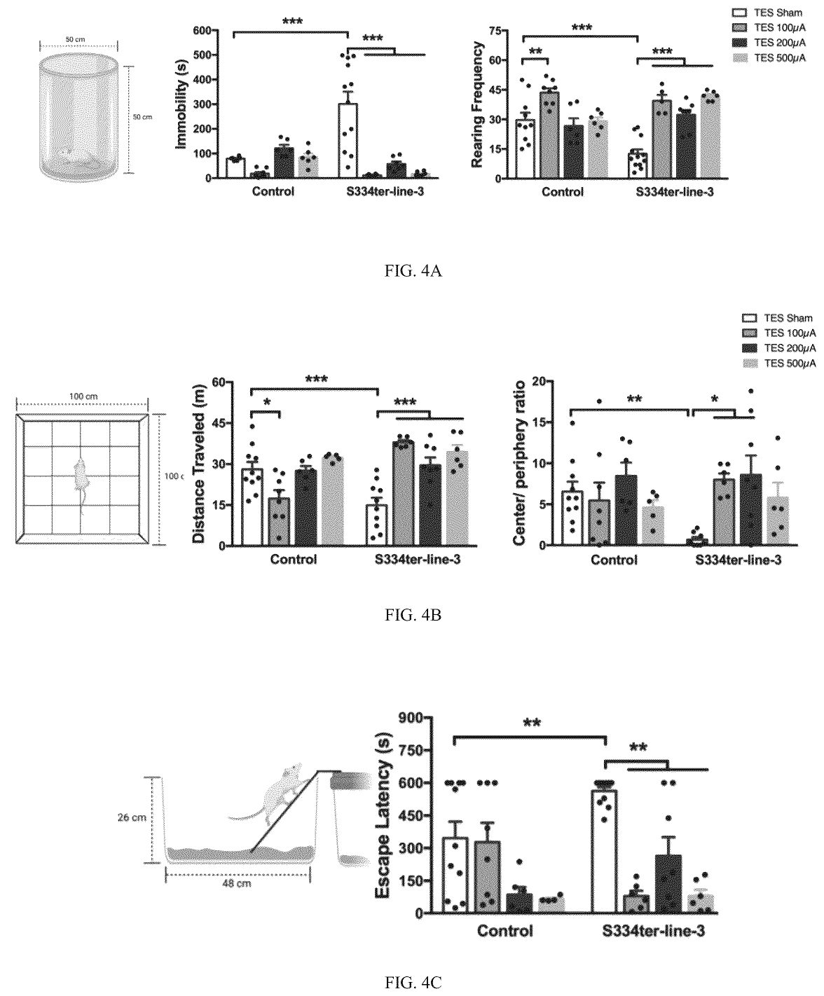

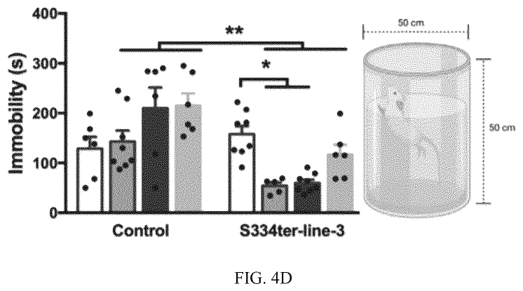

In the cylinder test (FIG. 4A), two-way ANOVA reveals significant effects of TES and TES x genotype on immobility time (TES: F_{3,49}=12.159, p<0.001; genotype: F_{1,49}=0.849, p=0.361; TES x genotype: F_{3,49}=10.014, p<0.001) and rearing frequency (TES: F_{3,53}=19.654, p<0.001; genotype: F_{1,53}=0.124, p=0.726; TES x genotype: F_{3,53}=11.065, p<0.001). The sham-treated S334ter-line-3 rats show an increased immobility time and a reduced rearing frequency when compared with sham-treated controls (ps<0.001), suggesting a reduction in locomotor activity and an increase in anxiety-like behavior. Administration of TES at 100, 200, and 500 μA effectively restores locomotor activity and induces anxiolytic behavior in S334ter-line-3 rats, as reflected by increased mobility and rearing behaviors (ps<0.001). Only TES at 100 μA increases rearing frequency in the control rats (p=0.003).

In the OFT (FIG. 4B), two-way ANOVA identifies significant effects of TES and TES x genotype on travel distance (TES: F_{3,53}=7.889, p<0.001; genotype: F_{1,53}=2.206, p=0.143; TES x genotype: F_{3,53}=16.402, p<0.001) and the ratio of time spent in center to periphery zones (TES: F_{3,49}=3.489, p=0.022; genotype: F_{1,49}=0.18, p=0.673; TES x genotype: F_{3,49}=2.874, p=0.046). The sham-treated S334terl-line-3 rats show a shorter distance traveled (p<0.001) and less time spent in the center zone (p=0.007) compared with sham-treated control rats, indicating a deficit in locomotor activity and an anxiety-like response in S334ter-line-3 rats, respectively. Treatment with TES at all tested amplitudes improves locomotion in S334ter-line-3 rats as seen by the increased distance traveled (ps<0.001), whereas only TES at 100 μA reduced the distance traveled in control rats (p=0.015). Furthermore, TES at 100 and 200 μA in S334ter-line-3 rats increase the time spent in center zone (100 μA: p=0.02; 200 μA: p=0.004), suggesting an anxiolytic effect.

In the HCET (FIG. 4C), two-way ANOVA shows significant TES and TES x genotype effects on escape latency (TES: F_{3,52}=13.379, p<0.001; genotype: F_{1,52}=0.959, p=0.332; TES x genotype: F_{3,52}=6.088, p<0.001). The sham-treated S334ter-line-3 rats show significantly longer escape latency compared with sham-treated control rats (p<0.002), indicating an anxiety-like response. Administration of TES at all the tested amplitudes significantly shortens the escape latency only in S334ter-line-3 rats (100 μA: p<0.001; 200 μA: p=0.003, 500 μA: p<0.001), which further suggests an anxiolytic effect induced by TES.

In the FST (FIG. 4D), two-way ANOVA indicates significant main effects for TES (F_{3,46}=3.105, p=0.036), genotype (F_{1,46}=25.489, p<0.001), and TES x genotype (F_{3,46}=4.704, p=0.006) on immobility time. Differences between genotypes were observed among the TES-treated rats, with S334ter-line-3 rats showing significantly lower immobility time compared to control rats given the same TES treatments (TES 100 μA: p=0.01; TES 200 μA: p<0.001; TES 500 μA: p=0.006), indicating a lower behavioral despair in the TES-treated transgenic rats. Furthermore, S334ter-line-3 rats treated with TES at 100 and 200 μA showed significantly decreased immobility time compared with the sham-treated rats (100 μA: p=0.019; 200 μA: p=0.01), suggesting a decrease in despair-like behavior after TES.

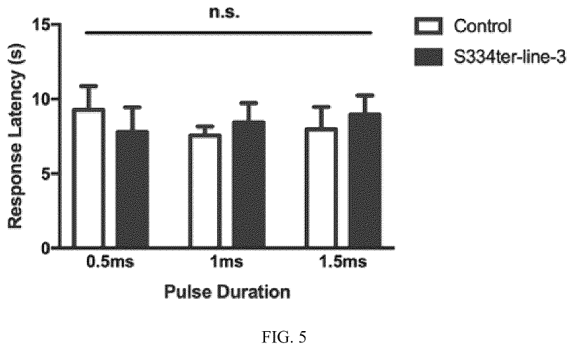

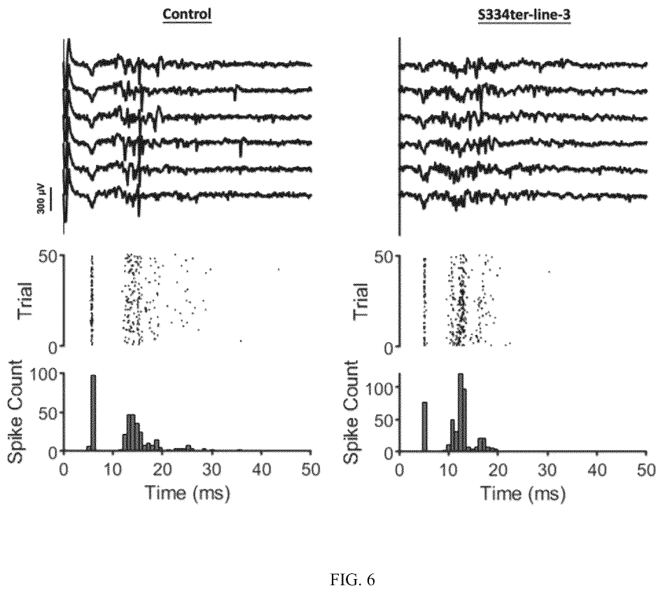

Electrically evoked potentials (EEPs) elicited by direct retinal stimulation are measured in the superior colliculus of S334ter-line-3 and control rats, showing that functional connectivity of the visual pathway is preserved in S334ter-line-3 rats. Two-way ANOVA shows no significant effects of genotype and pulse duration on the response latency of neurons (genotype: F_{1,21}=0.015, p=0.903; pulse duration: F_{2,21}=0.069, p=0.934; genotype x pulse duration: F_{2,21}=0.504, p=0.611; FIG. 5). EEPs in both S334ter-line-3 and control rats (FIG. 6) indicating that the visual pathways from retina to superior colliculus are activated in both transgenic and control rats. The EEP response windows are observed from 5 to 20 ms and consisted of early responses and late responses. The early responses occur at 5-8 ms and the late responses occur at 10-20 ms within the 25 ms response window after stimulation. These results suggest that the functional connectivity through the visual pathway is preserved in the transgenic rats.

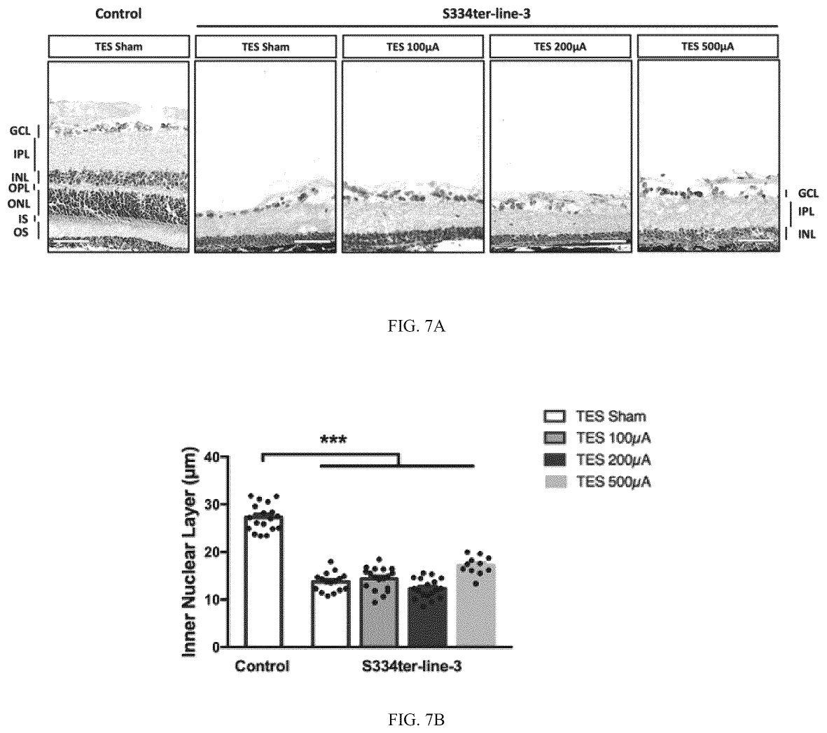

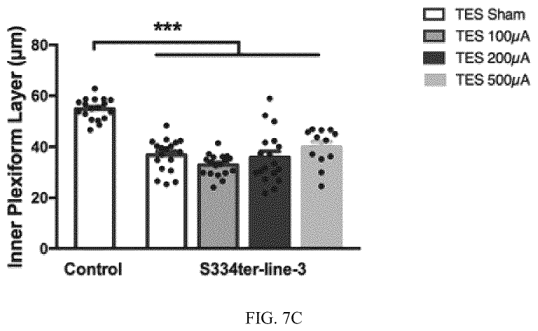

However, TES does not rescue the degenerating photoreceptor layer in S334ter-line-3 rats. In S334ter-line-3 rats, there are reduced retinal thickness and loss of outer plexiform layer, outer nuclear layer, and inner and outer segments of photoreceptors compared with control rats (FIG. 7A). One-way ANOVA reveals a significant group difference in the thickness of the inner nuclear layer (F_{4,80}=121.137, p<0.001; FIG. 7B) and inner plexiform layer (F_{4,83}=28.714, p<0.001, FIG. 7C). The mean thicknesses of the inner nuclear layer and inner plexiform layer are significantly reduced in S334ter-line-3 rats regardless of TES treatments (ps<0.001).

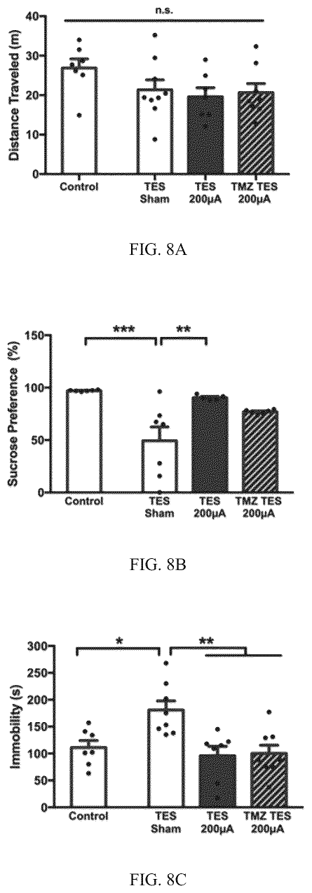

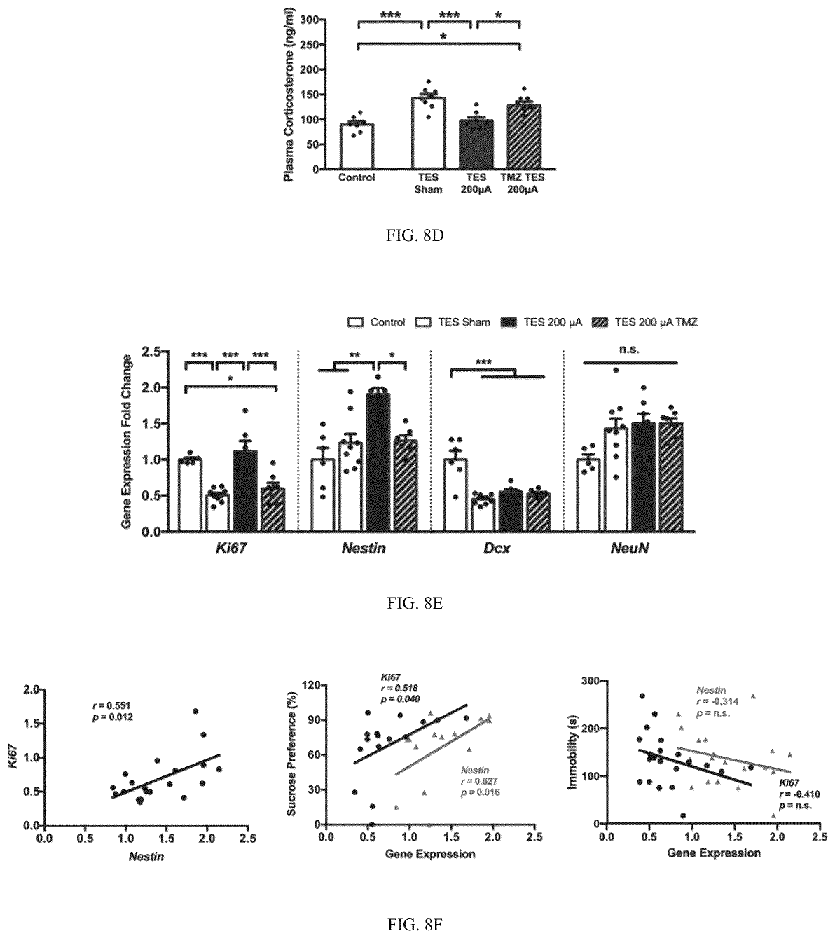

Further, it is proved that TES induce antidepressant-like behaviors in the CUS rat model. The effects of TES are further investigated in a CUS rat model of depression. In the OFT (FIG. 8A), one-way ANOVA shows no significant group differences in the distance traveled (F_{3,27}=2.613, p=0.074).

In the SuPT (FIG. 8B), one-way ANOVA identifies a group difference in the sucrose preference (F_{3,23}=7.765, p=0.001). The sham-treated CUS rats have significantly reduced sucrose preference compared with the non-CUS control rats (p=0.001), indicating an anhedonic-like response in the CUS rats. Treatment with TES at 200 μA effectively restores the reduced sucrose preference in the CUS rats compared to sham-treated rats (p ¼ 0.01), indicating a hedonic-like effect induced by TES. Such an effect is not observed in the TMZ+TES-treated rats, implying hedonic effect of TES is via a neurogenesis-dependent mechanism.

In the FST (FIG. 8C), one-way ANOVA shows a significant group difference in immobility time (F_{3,29}=6.491, p=0.002). The sham-treated CUS rats show higher immobility compared with the non-CUS rats (p=0.029), indicating an increase in behavioral despair in the CUS rats. The TES-treated rats with or without TMZ injection show significantly lower immobility time (non-TMZ: p=0.005; TMZ: p=0.006), suggesting the anti-despair-like effects of TES are via a neurogenesis-independent mechanism.

TES also reduces plasma corticosterone levels in the CUS rat model. The effects of CUS and TES on the level of corticosterone, a major stress hormone (FIG. 8D), are determined. One-way ANOVA reveals a significant group difference in the plasma corticosterone level (F_{3,29}=11.562, p<0.001). The sham-treated CUS rats have a significantly higher concentration of plasma corticosterone than non-CUS control rats (p<0.001), indicating a higher stress level due to CUS. Treatment with TES effectively reduces the corticosterone level in CUS rats (p=0.001). Treatment with TMZ+TES increases corticosterone level compared with TES alone (p=0.046) to a level comparable to the sham-treated group.

Further, TES upregulates neurogenesis-related markers in the CUS rat model. The gene expression of neurogenesis-related markers in the hippocampus of CUS rats is quantified by real-time PCR (FIG. 8E). One-way ANOVA identifies significant group differences in the gene expression of Ki67 (F_{3,26}=14.778, p<0.001), Nestin (F_{3,25}=7.935, p=0.001), and Dcx (F_{3,25}=15.684, p<0.001), but not NeuN (F_{3,27}=2.943, p=0.053). The sham-treated CUS rats show lower gene expression of Ki67 (p=0.001) and Dcx (p<0.001) compared with non-CUS rats. Treatment with TES effectively restores the expression of Ki67 (p<0.001). Moreover, the TES-treated CUS rats show a higher expression of Nestin compared with both the sham-treated CUS rats and non-CUS rats. Treatment with TMZ abolishes the upregulated expression of Ki67 and Nestin in the TES-treated CUS rats (Ki67: p=0.001; Nestin: p=0.018).

A correlation analysis is conducted to examine the association between the TES-induced antidepressant-like effects and neurogenesis-related markers in the CUS rats (FIG. 8F). There is a positive correlation between Ki67 and Nestin (r=0.551, p=0.012), suggesting they are closely associated with hippocampal neurogenesis induced by TES. Additionally, there are positive correlations between sucrose preference and gene expression of Ki67 (r=0.518, p=0.040) and Nestin (r=0.626, p=0.016), suggesting a positive association between neurogenesis and the hedonic-like effects of TES. On the other hand, there are no correlations between immobility time in the FST and Ki67 gene expression (r=−0.314, p=n.s.) or Nestin gene expression (r=−0.410, p=n.s.), which agrees with the behavioral results that showed neurogenesis-independent anti-despair effect of TES.

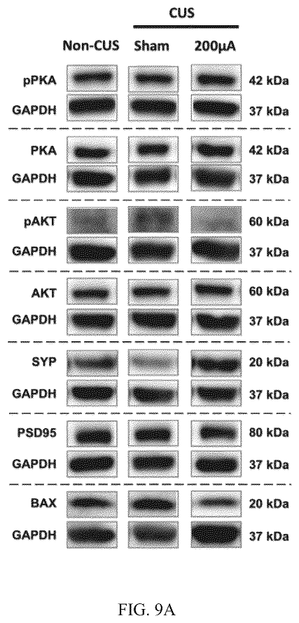

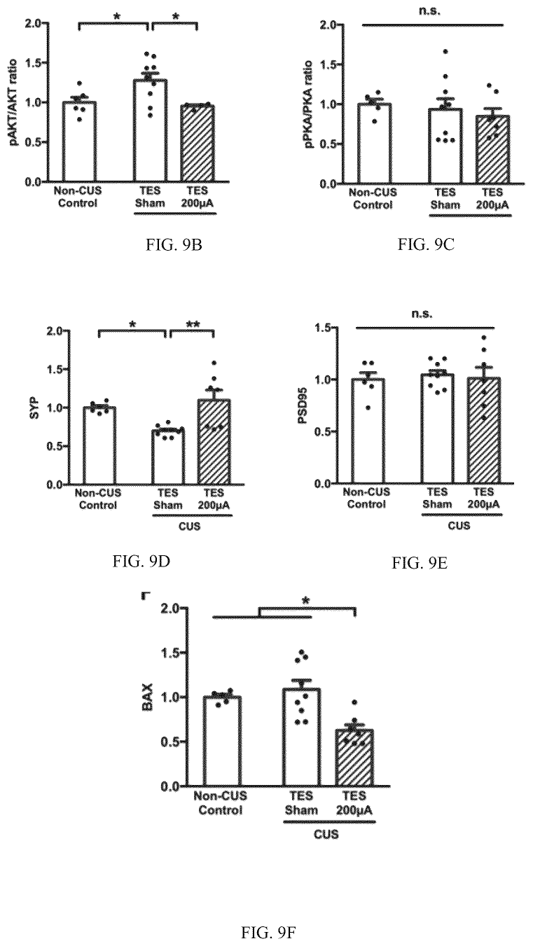

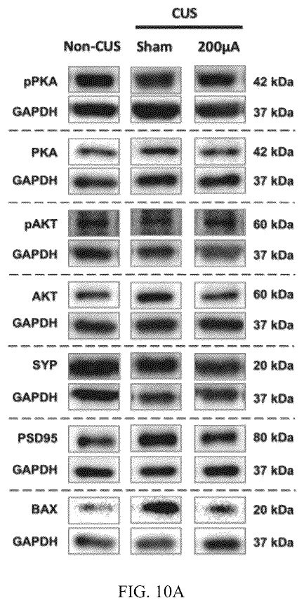

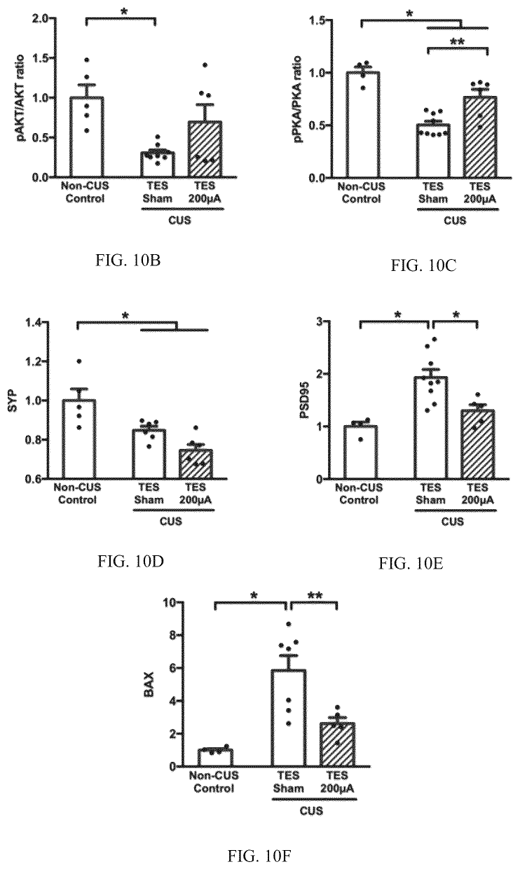

Furthermore, TES alters the expression of neuroplasticity-related proteins in the CUS rat model. Neuroplasticity changes induced by chronic stress are closely associated with the pathophysiology of depression. The effects of TES on neuroplasticity, particularly on the synaptic and apoptotic components are examined. To this end, neuroplasticity-related proteins in the hippocampus (FIG. 9A) and amygdala (FIG. 10A) of the CUS model are evaluated. In the hippocampus, one-way ANOVA shows significant group changes in pAKT/AKT ratio (F_{2,18}=8.136, p=0.004; FIG. 9B), pPKA/PKA ratio (F_{2,20}=0.357, p=0.704; FIG. 9C), SYP (F_{2,21}=8.172, p=0.003; FIG. 9D), PSD95 (F_{2,21}=0.111, p=0.895, FIG. 9E), and BAX (F_{2,20}=8.283, p=0.003; FIG. 9F). The sham-treated CUS rats show a higher pAKT/AKT ratio (p=0.013) and a lower SYP expression (p=0.036) compared with the non-CUS control rats, whereas TES treatment reverses these changes (pAKT/AKT: p=0.013; SYP: p=0.003). Moreover, TES treatment also reduces BAX expression compared with the non-CUS control rats (p=0.038) and sham-treated rats (p=0.003).

In the amygdala, one-way ANOVA shows significant group changes in pAKT/AKT ratio (F_{2,18}=6.097, p=0.011; FIG. 10B), pPKA/PKA ratio (F_{2,18}=28.687, p<0.001; FIG. 10C), SYP (F_{2,16}=11.354, p=0.001; FIG. 10D), PSD95 (F_{2,17}=10.422, p=0.001; FIG. 10E), and BAX (F_{2,15}=11.624, p=0.001; FIG. 10F). The sham-treated CUS rats have lower PAKT/AKT ratio (p=0.011), pPKA/PKA ratio (p<0.001), and SYP level (p=0.039), but elevated PSD95 (p=0.002) and BAX expression (p=0.002). Treatment with TES effectively reverses the CUS-induced changes on pPKA/PKA ratio (p=0.006), PSD95 (p=0.023) and BAX (p=0.019). Together, these results suggest that TES induces neuroplasticity-related effects in the CUS model, potentially leading to improvements in synaptic plasticity and reduced apoptosis in the hippocampus and amygdala.

In summary, patients with visual impairment are more vulnerable to developing depression and anxiety. To investigate whether the S334ter-line-3 rat model of retinal degeneration can develop depressive-like behaviors similar to depressive symptoms in humans, a series of behavioral tests mimicking the clinical diagnosis of depression is conducted. The S334ter-line-3 rats exhibit elevated anxiety levels, as demonstrated in the cylinder test, OFT, and HCET. There are no significant differences in the forced swim immobility time between the sham-treated S334ter-line-3 and control rats, which suggests the retinal degeneration model did not exhibit increased despair compared to control rats.

The electrophysiology study in S334ter-line-3 rats shows the integrity of visual pathway from the retina to superior colliculus is preserved. The superior colliculus is a primary subcortical relay for visual information. An intact visual pathway can potentially allow the transmission of TES-induced input to visually connected brain regions, such as the limbic system. In S334ter-line-3 rats, TES significantly ameliorates anxiety-like behaviors as indicated by the cylinder test, OFT, and HCET. Surprisingly, despite the lack of despair-like behavior in the sham-treated S334ter-line-3 rats, TES at 100 and 200 μA significantly decrease immobility time in the FST, implying an anti-despair-like effect of TES. In the control rats, TES does not generally lead to pronounced behavioral changes, except TES at 100 μA, which increases rearing in the cylinder test and decreases the distance traveled in the OFT. Overall, the effects of TES on control rats are inconclusive, as TES at 200 and 500 μA does not induce significant changes in all anxiety- and depression-related behavioral parameters.

Comparing the effects of different stimulation amplitudes, it is found that TES at 100 and 200 μA elicit the most profound anxiolytic effects in S334ter-line-3 rats in all behavioral tests, whereas TES at 500 μA fails to induce significant improvements in the OFT and FST. These findings are in agreement with a study investigating the optimal neuroprotective parameters of TES, which shows repeated administration of 30-60 min of TES at 100 or 200 μA amplitude, 1 or 2 ms pulse width, and 20 Hz frequency induces the most beneficial effects by enhancing the survival of axotomized retinal ganglion cells in rats. Therefore, TES at a moderate intensity of 100 or 200 μA is optimal for relieving ophthalmological or mental disorders in rats.

The retinal histology between the TES and sham-treated groups are compared to determine whether TES can rescue the degenerated retinal layers in S334ter-line-3 model. It is found that S334ter-line-3 rats have significantly thinner retinas with a loss of photoreceptor layers compared to age-matched controls. The histological assessment of degenerated retinas shows that TES at all tested amplitudes is unable to rescue the atrophied retinas. Given the inherent progressive retinal degeneration in S334ter-line-3 rats, the TES treatment in the present invention might not be optimal or fall within the therapeutic window to rescue the retinas, as the TES treatment was started at 10 weeks. Another possible explanation is that the retinas are already too deteriorated before TES is applied. As the retinal thickness is generally correlated with a decline in retinal and visual functions, the results imply that TES does not enhance visual ability in S334ter-line-3 rats, and therefore the behavioral improvements observed are mediated through non-visual components.

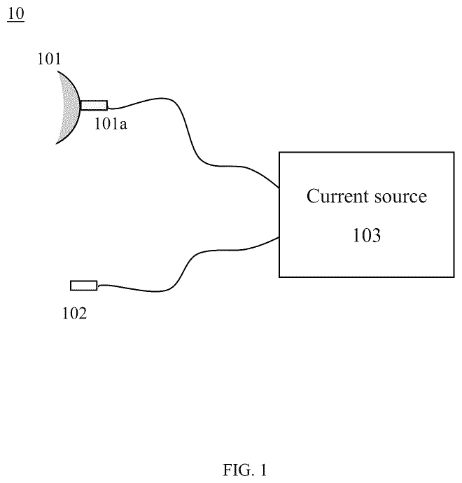

Due to the promising behavioral results in the retinal degeneration model, the antidepressant-like effects and the underlying mechanisms of TES are further evaluated using the CUS rat model. The TES treatment is applied at 200 μA based on its promising antidepressant-like effects in S334ter-line-3 rats (FIG. 1), and on previous findings that identified TES at 200 μA is more potent than at 100 μA in promoting the expression of neurotrophins and normalizing apoptotic factors. It is shown that TES at 200 μA counteract the CUS-induced anhedonia- and despair-like responses, which are the core symptoms of depression in humans. Although TES is shown to alter locomotor activity in S334ter-line-3 rats, a comparable locomotor ability in the CUS model regardless of the TES treatment as measured by the distance traveled is observed, which should eliminate any confounding effects of locomotion on the behavioral performance.

It is widely recognized that chronic stress disrupts neuroplasticity and plays a vital role in the development of depression, whereas antidepressants exert opposite effects by enhancing neuroplasticity. The effects of TES are assessed on different aspects of neuroplasticity, including neurogenesis, synaptic plasticity, and apoptosis. The gene expression results suggest that the antidepressant-like effects of TES may involve a neurogenesis mechanism. Treatment with TMZ abolishes TES induced hedonic-like effect, but not the anti-despair-like effect, suggesting that TES induces both neurogenesis-dependent and -independent effects, respectively. The neurogenesis-dependent hedonic effect of TES is supported by the positive correlations between sucrose preference and the expression of Ki67 and Nestin genes. On the other hand, the neurogenesis-independent anti-despair-like effect of TES is supported by the lack of correlation between forced swim immobility time and Ki67 or Nestin expression. However, it is worthy to note that the use of TMZ as a blocking agent for cell proliferation not only inhibits neurogenesis, but may also suppress proliferating glial cells. The results cannot eliminate the involvement of a gliogenic effect by TES. In particular, ki67 and nestin are expressed in both neuronal and glial progenitor cells. Although nestin expression is typically restricted to neuronal progenitors under physiological conditions, previous research showed that neuroinflammatory insults, including under chronic stress, could increase microglial proliferation and shift nestin expression towards activated microglia/macrophages. This might suggest that nestin could act as a marker of inflammatory responses. Nevertheless, the expression of Ki67 and Nestin reported in the present invention are unlikely to be derived from neuroinflammation or microglial proliferation as an increase in Ki67 and Nestin after CUS is not observed. Furthermore, earlier findings demonstrated an anti-inflammatory role of TES through suppressing microglial activation. The upregulation of Ki67 and Nestin expression after TES treatment in the present invention favors a neurogenic effect instead of a glial-mediated inflammatory effects. However, further studies are needed to investigate whether glial cells or gliogenesis play a role in the antidepressant-like effects of TES.

Depressed patients and animal models consistently show a dysfunctional hypothalamic-pituitary-adrenal (HPA) axis, as shown by elevated circulating glucocorticoid levels. On the other hand, antidepressant drugs can also normalize glucocorticoid secretions. To this end, the plasma corticosterone level in the CUS rats is investigated, which shows higher plasma corticosterone indicating a state of stress and dysregulated HPA axis. Treatment with TES effectively attenuates the stress-induced rise in corticosterone levels, suggesting the antidepressant-like effects of TES might involve normalization of the HPA axis. It has been reported that animals with depleted neurogenesis by TMZ or radiation treatments have higher corticosterone levels, likely due to the loss of a stress response buffering system in the newly formed hippocampal granule neurons. However, in the current study, the TMZ+TES-treated CUS rats does not show significantly higher corticosterone levels compared to sham-treated CUS rats, suggesting that TES can reduce corticosterone levels regardless of the presence of a neurogenesis blocker.

In depression, stress is reported to alter synaptic plasticity and apoptosis in the hippocampus and amygdala. The hippocampus and amygdala have functional connections with the cortical visual pathway and subcortical retinotectal pathway, and hence, are also implicated in the processing of visual emotional stimuli. In the present invention, it is identified that synaptic dysfunctions occur in the CUS model, as demonstrated by a significant decrease in the presynaptic marker SYP in both the hippocampus and amygdala, and an increase in the postsynaptic marker PSD95 in the amygdala. It is found that TES in the CUS rats effectively normalized the expression of SYP and PSD95 in the hippocampus and amygdala, respectively, suggesting that TES may induce a synaptic effect that contributes to the antidepressant-like responses. Moreover, the expression of a pro-apoptotic marker Bax is downregulated by TES in the CUS animals, suggesting a potential anti-apoptotic mechanism of TES. Both AKT and PKA signaling pathways are implicated in neuroplasticity. The kinase AKT regulates synaptic plasticity and neuronal apoptosis and is widely expressed in emotional circuits, including the hippocampus and amygdala. Although AKT activity is found to be decreased by stress and depression, various stress models have reported the opposite, with higher pAKT levels found in the hippocampus and amygdala. In the present invention, the CUS rats exhibit increased hippocampal AKT activity, which is normalized by TES treatment, suggesting the beneficial effects of TES may involve the modulation of AKT signaling. In addition, reduced PKA activity is observed in the amygdala, as indicated by a lower pPKA/PKA ratio. As a major regulator of many cyclic adenosine monophosphate-dependent biological processes, PKA plays a pivotal role in neuronal survival and synaptic plasticity. Reduction of PKA in the amygdala has been demonstrated in a social defeat stress model of depression and postmortem brains of depressed patients. Not surprisingly, antidepressants can enhance PKA activation in the amygdala of rats. Furthermore, anxiety-like behaviors in the social defeat stress model are alleviated by a PKA activator and aggravated by a PKA inhibitor administered in the amygdala. The present invention shows that TES restores PKA activity in the amygdala of CUS model agrees with the abovementioned studies, suggesting a role of PKA signaling in the amygdala on the effects of TES.

In summary, the above results evidence that TES can induce antidepressant-like effects in S334ter-line-3 and CUS rats. Especially, the results from the CUS model highlight the antidepressant-like effects of TES possibly act through the normalization of plasma corticosterone and modulation of neuroplasticity within the hippocampus and amygdala involving neurogenesis, synaptic plasticity, and neuronal apoptosis.

### Example 2. Evaluating the Effect of TES on Enhancing Cognitive Functions in Aged and 5XFAD Mouse Model

The effects of TES on memory functions are evaluated in both aged and 5XFAD mice by the Y-maze forced alternation and Morris water maze (MWM) tests.

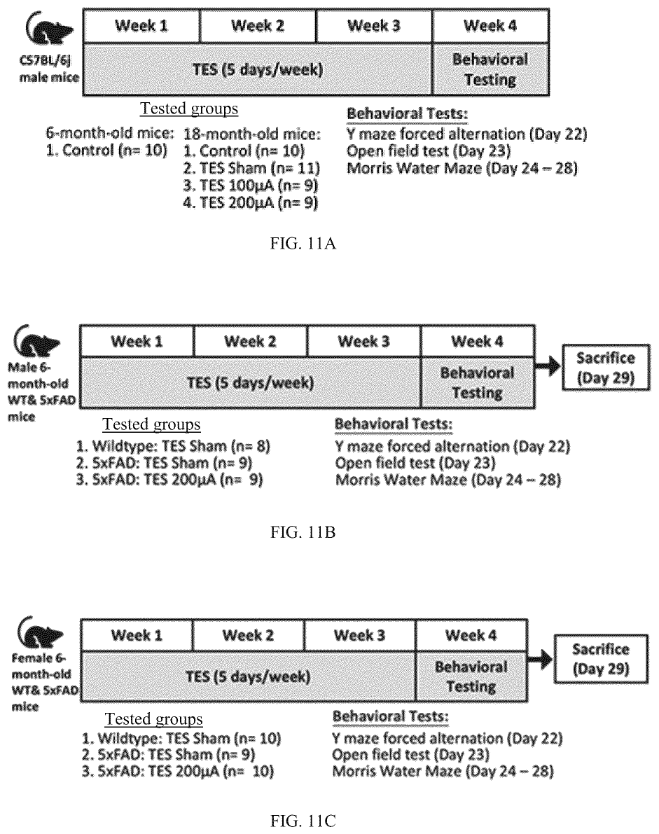

In the aged mice (FIG. 11A), behavioral comparisons are made between young (n=10) and aged (n=10) mice, and between aged mice receiving either TES sham (n=11), TES at 100 μA (n=9), or TES at 200 μA (n=9). In the 5XFAD model, memory functions are assessed separately for male (FIG. 11B) and female (FIG. 11C) wild-type mice (males: n=8, females n=10) and 5XFAD mice receiving TES sham (males: n=9, females: n=9), or TES at 200 μA (males: n=9, females: n=10). Following behavioral testing, 5XFAD mice are sacrificed and brains are harvested for β-amyloid staining and protein expression study to investigate the effects of TES on plaque deposition and synaptic components.

For receiving the TES treatment, Mice are anesthetized with 1-3% isoflurane during the procedure of stimulation. A pair of stimulating electrode rings made from stainless steel is placed on the corneas and connected to a digital stimulator via a stimulus isolator. The impedance of stainless-steel electrodes is 112±4.2Ω at 1 kHz and 85.1±1.8Ω at 100 kHz. Eye moisturizing gel is applied to prevent dehydration. A pair of car clip electrodes is used as the reference electrodes. Electrical current (frequency: 20 Hz, pulse width: 1 ms, amplitude: 100 or 200 μA) is delivered 1 h daily, 5 days per week for 3 weeks. Sham groups are similarly treated but with the stimulator turned off. The stimulation parameters are determined based on previous studies that demonstrates the neuroprotective effects of TES in animal models. Control groups are also subjected to 1 h of isoflurane anesthesia on stimulation days to eliminate confounding effects of isoflurane exposure on cognitive performance.

The effects of TES on cognitive performance are evaluated in the Y-maze forced alternation and MWM tests, whereas effects on locomotion and anxiety are evaluated in the open field test (OFT). Since anxiety can disrupt cognitive performance, a similar anxiety level across tested groups is needed for fair comparisons in memory tests. All behavioral tests are performed during the dark cycle by researchers blinded to the treatments and testing conditions. All tests are video recorded and scored using the ANY-maze behavioral tracking software.

The Y-maze apparatus consists of three identical arms (L: 35×W: 6×H: 10 cm³) made of white plastic positioned in a Y shape. This test assesses the rodents' spatial recognition memory, which relies on their propensity to explore novel environments. During the acquisition phase, one arm is designated as the novel arm and is blocked. Mice are placed at the end of the start arm facing away from the center and allowed to inspect the two open arms for 5 min. After a 30 min retention period, the retrieval phase is performed with all arms opened. Again, mice are placed at the end of the start arm and allowed to explore all arms of the Y-maze for 5 min. The frequency of visits to the novel arm is analyzed.

The OFT is performed to assess anxiety and locomotor activity in mice. The OFT is conducted in a white Plexiglas box (40×40×40 cm³) with an open top. Mice are individually placed in the center of the open field and allowed to explore freely for 10 min. The total distance traveled, and time spent in center and periphery zones are analyzed.

The MWM test is conducted in a black circular pool (diameter: 116 cm, height: 28 cm) filled with water at 25±1° C. to a height of 15 cm. The water is made opaque by adding 0.5 kg of skimmed milk powder. A transparent escape platform (diameter: 10 cm) is placed 1.5 cm below the water and 10 cm away from the sidewall. During the training phase, mice are trained to locate the hidden platform in four trials daily for 4 consecutive days. In each trial, mice are released into the water facing the wall at one of the four starting positions opposite to the target quadrant where the platform is located. Mice are allowed to stay on the platform for 15 s after locating the platform within 1 min; otherwise, they are gently guided onto the platform and allowed to stay for 15 s. The escape latency is recorded for each trial. At 24 h following the last training session, the probe test is conducted with the platform removed from the pool. Each mouse is released to the opposite quadrant and allowed to explore the pool for 1 min. The time spent in the target quadrant and frequency of crossing the imaginary platform are analyzed.

The histological study is performed for β-amyloid evaluation. One day after the last behavioral test, mice are deeply anesthetized with sodium pentobarbital and transcardially perfused with cold Tyrode solution, followed by 4% paraformaldehyde. Brain tissue is dissected and post-fixed in the same fixatives overnight. Tissues are cryoprotected in 15% and 30% sucrose in 0.01 M PBS, and then snap-frozen in liquid nitrogen. Coronal sections (20 μm thickness) of the hippocampus are obtained on a cryostat. The sections are immunostained for β-amyloid using the avidin-biotin-peroxidase complex (ABC) method. In brief, free-floating sections are blocked with 0.5% H₂O₂ solution and 1% BSA and then incubated with mouse anti-β-amyloid primary antibody overnight at 4° C. Sections are washed and incubated with biotinylated goat anti-mouse IgG (1:1000) for 2 h at room temperature. To amplify target antigen signals, sections are incubated in ABC reagent for 2 h. Immunoreactivity is visualized in a solution containing 3,3′-diaminobenzidine tetrahydrochloride, 0.01% H₂O₂ solution, and nickel chloride. Finally, sections are mounted on slides and counterstained with hematoxylin.

Amyloid burden in the hippocampus is quantified by immunohistochemical analysis using a microscope equipped with a camera and image processing software. Hippocampal subregions, including the dentate gyrus, CA1/2, CA3, and subiculum, are delineated according to the Franklin and Paxinos mouse atlas (dorsal hippocampus: Bregma −1.3 to −2.4 mm, ventral hippocampus: Bregma −2.9 to −3.4 mm). Images of the hippocampus are taken under 5× magnification and then converted to 8-bit black and white images. Threshold intensity is adjusted to remove background noise before quantifying the percentage area covered by the stained β-amyloid. Measurements are conducted on two to four hippocampal sections per mouse in three to five mice per group.

For Western blotting, briefly, the dorsal and ventral regions of the hippocampus are grossdissected from freshly frozen brain tissues, followed by homogenization in lysis buffer containing Halt protease and phosphatase inhibitor cocktails. Protein concentrations are measured by Bradford assay. Equal amounts of protein (15 μg) are separated by 10% SDS-PAGE and transferred to PVDF membranes. The membranes are blocked with 5% milk in TBS for 1 h at room temperature and then incubated with primary antibodies overnight at 4° C. Antibodies against PSD95 (1:1000), synaptophysin (SYP; 1:1000), ionized calcium binding adaptor molecule (IBA-1; 1:1000), glial fibrillary acidic protein (GFAP; 1:1000), and glyceraldehyde 3-phosphate dehydrogenase (GAPDH; 1:1000) are used. The membranes are subsequently incubated with corresponding horseradish peroxidase-conjugated goat anti-rabbit or anti-mouse (1:2000) for 2 h. The immunoreactive signals are visualized by Clarity Western ECL Substrate using an imaging system. Densitometry analysis of protein bands is performed using an image processing software. The relative protein expression is normalized against GAPDH.

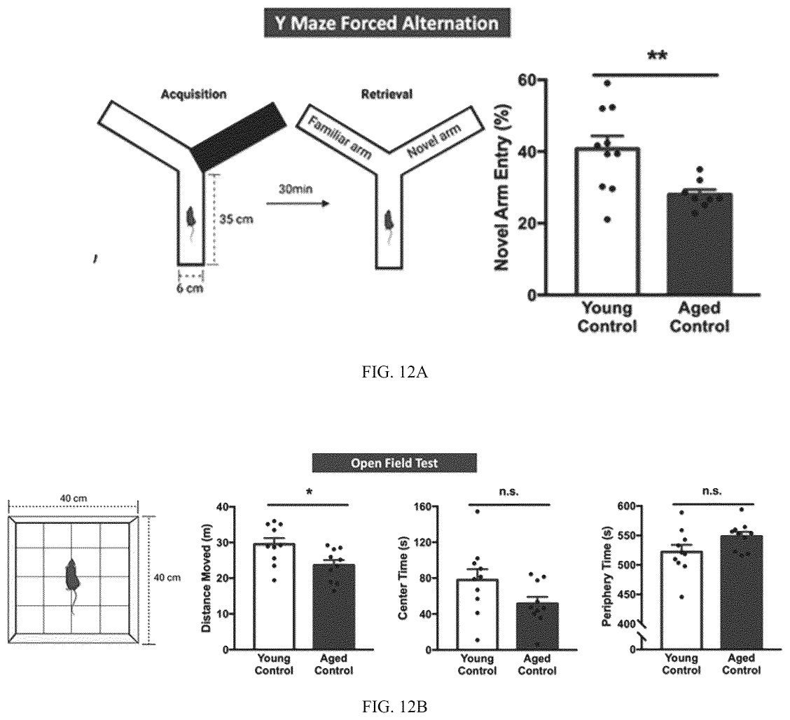

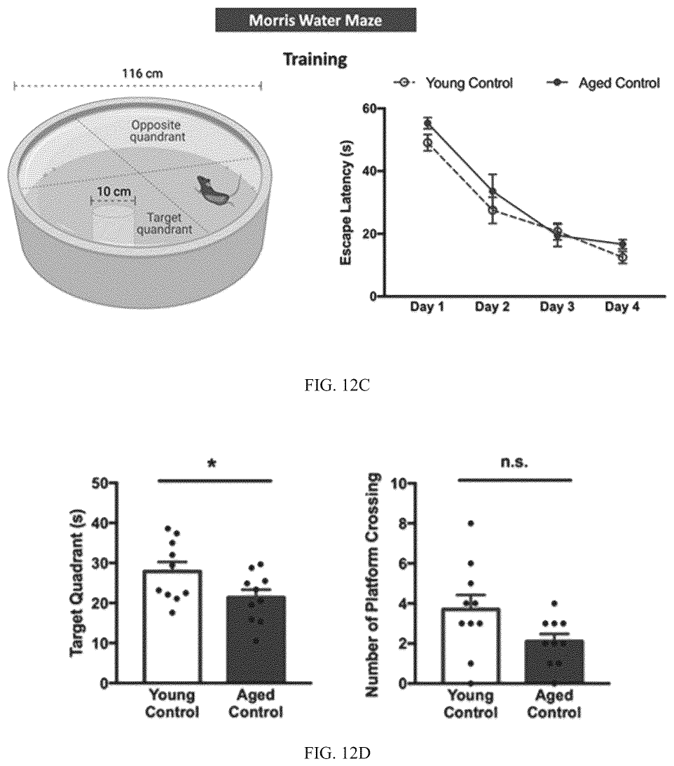

Memory functions and locomotor activity of aged mice are compared with that of young mice. In the Y-maze forced alternation test, aged mice exhibit impairments in spatial memory, as indicated by the significantly lower percentage of entry into the novel arm compared with young mice (FIG. 12A). In the OFT, aged animals show a decline in locomotor activity, as determined by the shorter distance traveled compared with young mice (FIG. 12B). Nevertheless, it is only observed that a trend toward altered anxiety-like behavior in older mice, as there are no significant differences in the time spent in the center or periphery zones between aged and young animals. The spatial learning and memory functions in the aged model are further examined. In the training phase of the MWM test, no differences are found between aged and young mice in the latency to locate the hidden platform (FIG. 12C). In the probe trial of the MWM test, aged mice spend less time in the target quadrant than young mice (FIG. 12D), further suggesting age-related cognitive deficits.

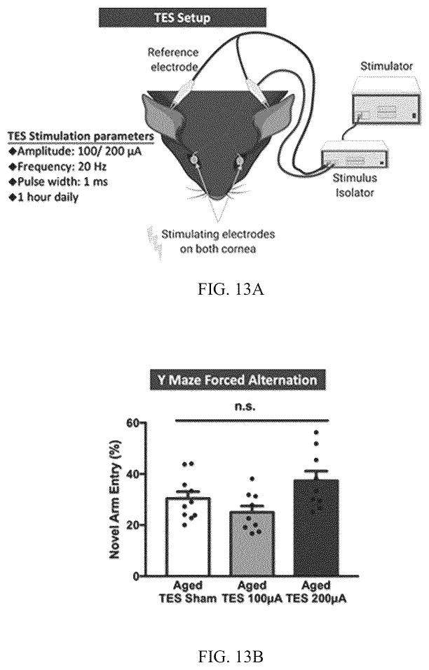

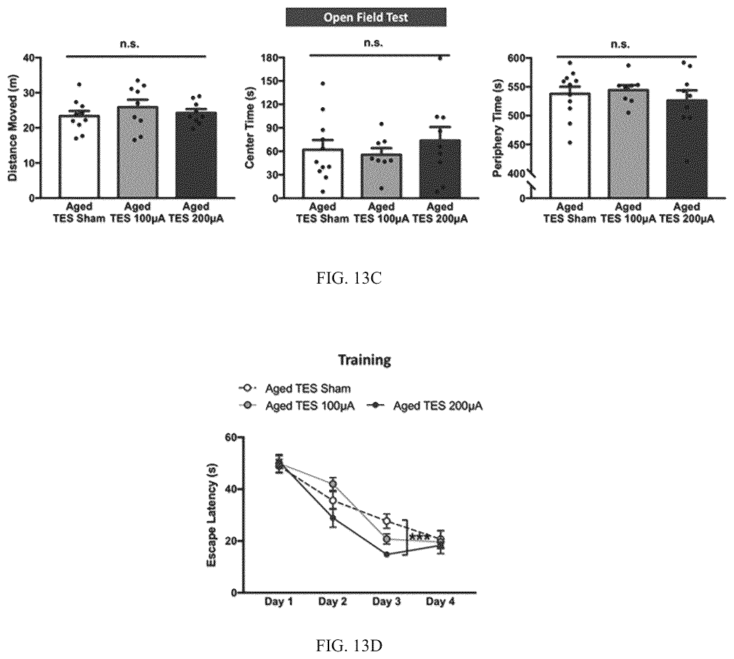

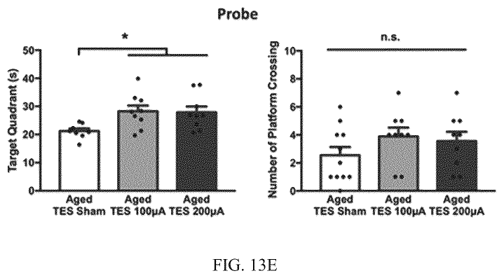

To investigate if TES restores the memory deficits, aged mice undergo chronic stimulation at either 100 or 200 μA prior to behavioral testing (FIG. 13A).

In the retrieval phase of the Y-maze test, TES at both stimulating amplitudes does not improve spatial memory in aged mice, with no significant changes in the preference for novel arm among the sham- and TES-treated groups (FIG. 13B). In the OFT, TES does not restore locomotor activity in aged animals, as there are no significant differences in the total distance moved (FIG. 13C). The TES-treated mice spend a similar amount of time in the center and periphery zones of the open field, indicating no changes to anxiogenic nor anxiolytic behavior in aged mice. In the MWM test, aged mice treated with TES at 200 μA have better performance in spatial learning, as shown by a shorter escape latency during the training phase (FIG. 13D). In agreement with the improvements observed in the learning phase, aged mice with TES at either 100 or 200 μA spend significantly longer time in the target quadrant than their sham-treated littermates, suggesting a restoration of spatial memory function in the TES-treated aged mice (FIG. 13E). Overall, prolonged TES treatment in aged mice does not improve memory function in the Y-maze test, whereas TES at both 100 and 200 μA ameliorate spatial memory deficits in the MWM probe test, and TES at 200 μA is sufficient to elicit improvement in spatial learning.

TES also improves spatial learning and memory in male 5XFAD mice. The effects of TES on memory functions are further investigated in 6-month-old male 5XFAD mice. TES conducted in aged mice identifies 200 μA as a more optimal TES amplitude for improving spatial learning and memory retrieval; hence, TES at 200 μA is applied in 5XFAD animals.

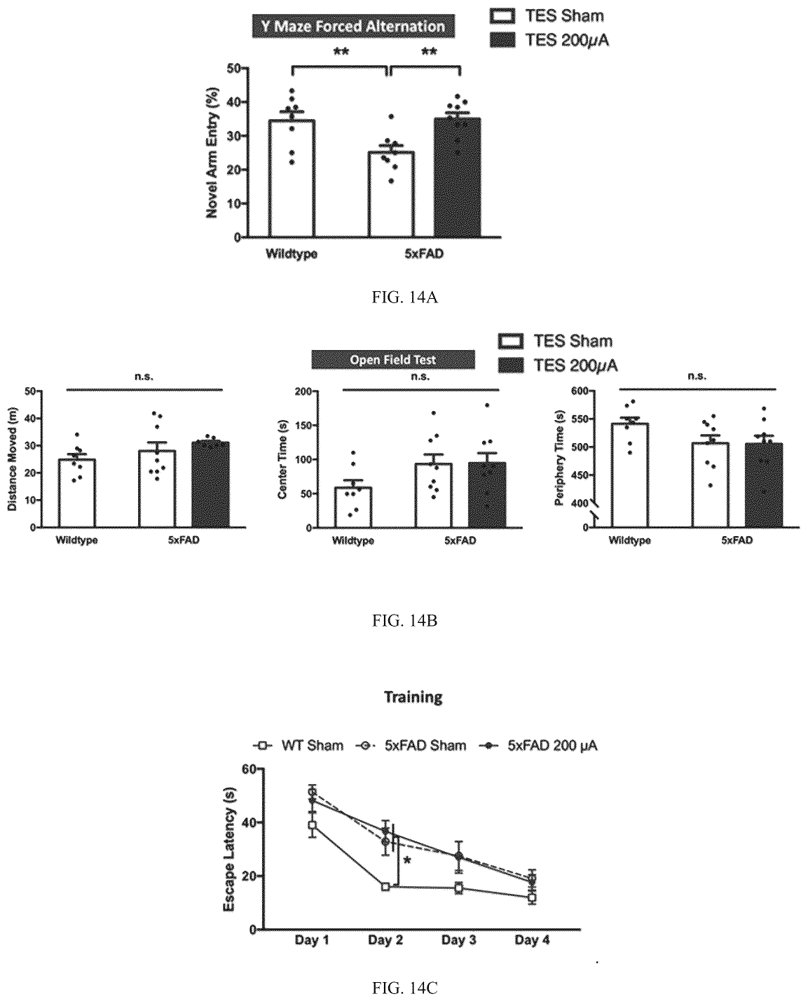

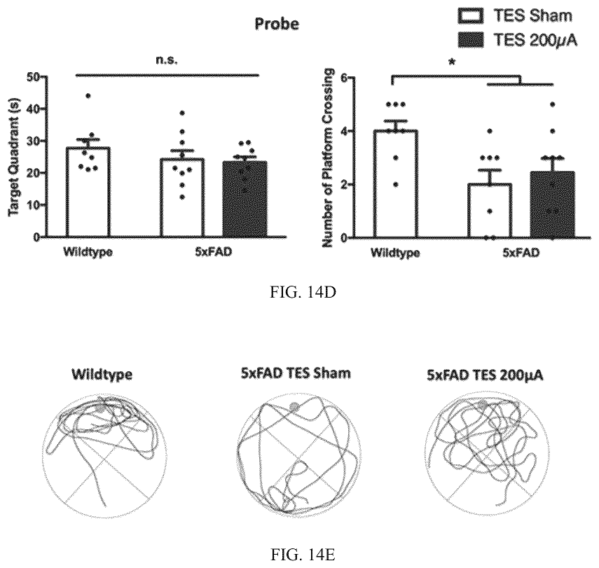

The state of cognitive decline is first validated in 5XFAD mice. The Y-maze test and OFT are conducted to compare spatial memory and locomotor ability in 6-month-old 5XFAD mice and their wild-type littermates. In the Y-maze test, 5XFAD mice show a significantly lower preference for the novel arm, compared with wild-type mice, confirming impaired spatial memory (FIG. 14A). TES at 200 μA effectively normalizes the performance in Y-maze, suggesting the restoration of spatial memory function. In the OFT, 5XFAD mice show locomotor activity and anxiety level comparable to their wild-type littermates, as indicated by the similar total distance traveled and time spent in the center or periphery zones, respectively (FIG. 14B). TES treatment does not alter their behavior in the OFT, suggesting that TES does not adversely affect locomotion or lead to anxiety-like behavior.

Memory functions are further evaluated by the MWM test. Although all groups can learn during the training days, 5XFAD mice exhibit a decline in learning ability, as demonstrated by a longer escape latency on day 2 of training compared with wild-type mice (FIG. 14C). Additionally, 5XFAD mice show a reduced number of crossings over the imaginary platform during the probe test, suggesting deficits in spatial memory retrieval (FIG. 14D and FIG. 14E). TES treatment in 5XFAD mice is unable to restore learning and memory functions in the MWM test. Overall, these results show the male 5XFAD mice presented with cognitive deficits, and long-term TES treatment effectively enhances spatial memory only in the Y-maze test without affecting general locomotor activity or anxiety level.

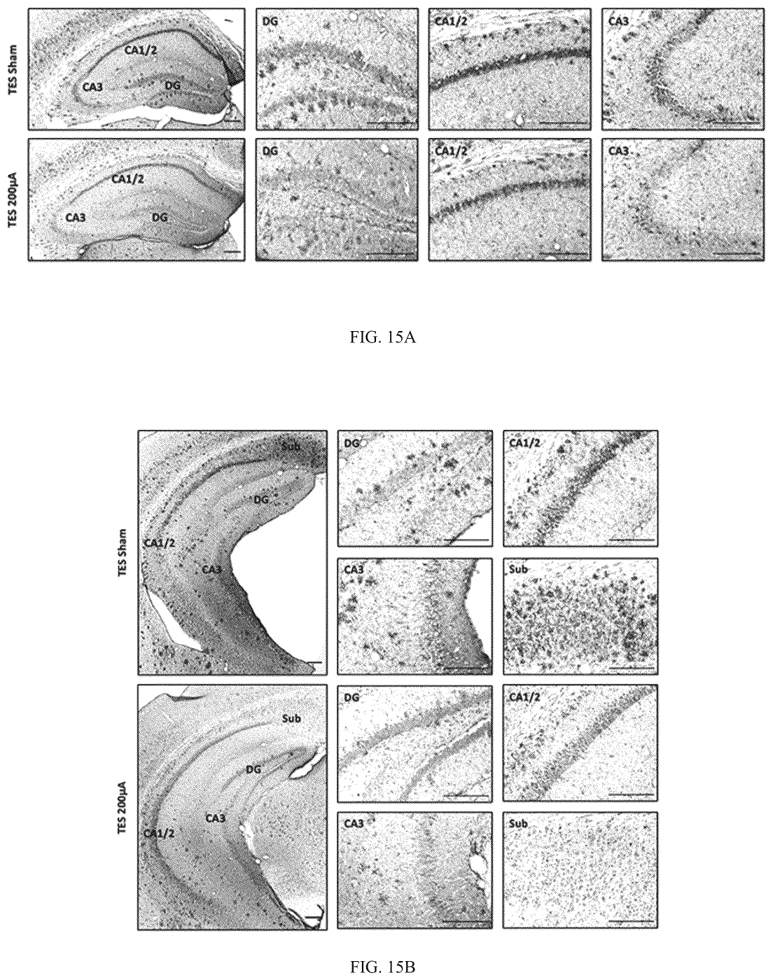

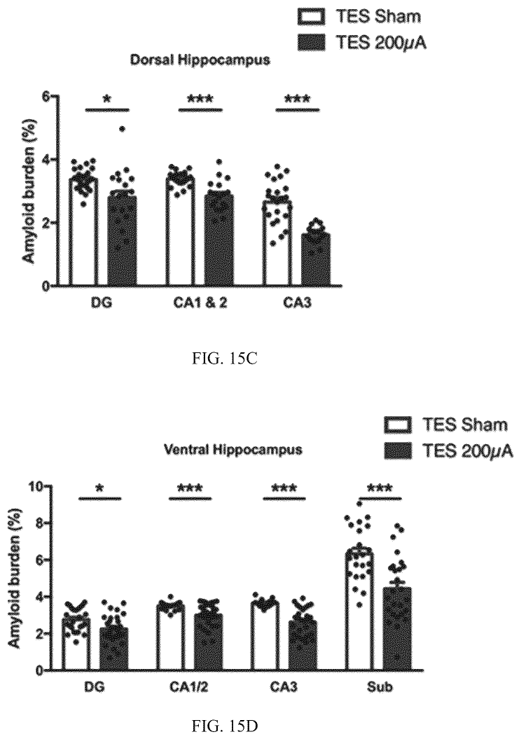

Further, TES ameliorates β-amyloid deposition in male 5XFAD mice. In AD, β-amyloid deposition is associated with cognitive decline. Previous studies reported that memory improvements in the 5XFAD model are accompanied by a reduction in amyloid burden. To evaluate the effect of TES treatment on plaque deposition in male 5XFAD mice, hippocampal sections from the sham- and TES-treated 5XFAD mice are stained with anti-β-amyloid (4G8) to assess plaque burden. β-amyloid deposition is evaluated in the DG and CA1 & 2, CA3, and subiculum regions in 5XFAD mice (FIGS. 15A-15B), which are significantly reduced by long-term TES administration (FIG. 15C-15D).

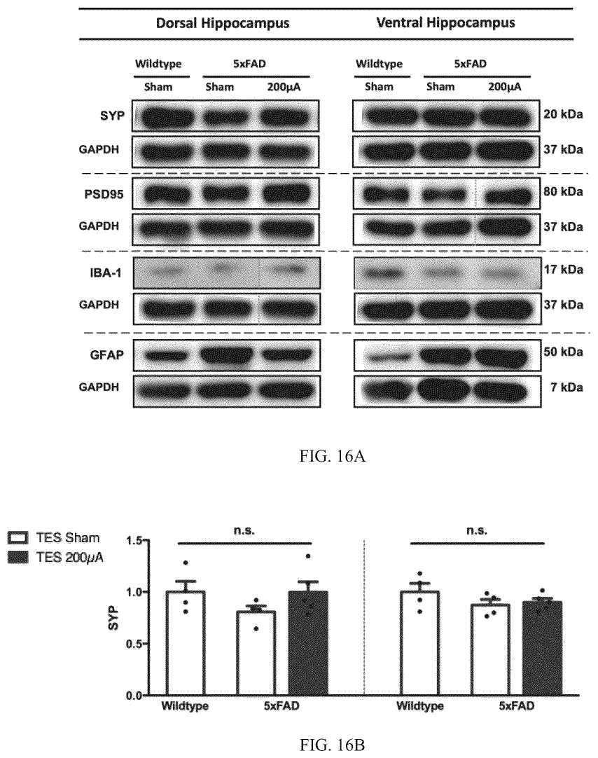

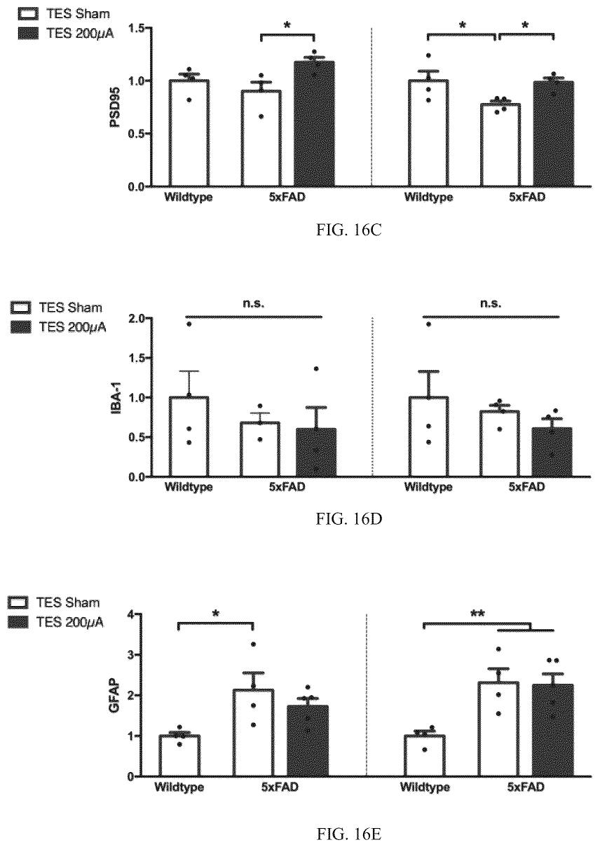

TES also upregulates hippocampal PSD95 expression in male 5XFAD. Mice. Synaptic dysfunction is strongly associated with cognitive decline and is widely recognized as a key feature in AD pathogenesis. To examine the effects of TES on synaptic components potentially involved in the observed memory improvements, protein expression levels of SYP and PSD95, which are pre- and postsynaptic markers, respectively, are examined (FIG. 16A). The unaltered SYP level (FIG. 16B) and significantly reduced PSD95 expression are found in the sham-treated 5XFAD mice, compared with wild-type controls, indicating postsynaptic deficits in the hippocampus of 5XFAD males (FIG. 16C). Treatment with TES increases PSD95 expression in both the dorsal and ventral hippocampi, suggesting the effects of TES involved a postsynaptic mechanism.

Furthermore, it is well established that the degeneration of synapses and neurons in AD progression is mediated by neuroinflammation through the functions of microglia and astrocytes. To this end, the effects of TES on glial-mediated inflammation is assessed by measuring the expression of IBA-1 and GFAP, which are the markers of reactive microglia and astrocytes, respectively (as shown in FIG. 16A). Although there are no significant changes observed in IBA-1 expression (FIG. 16D), it is found that GFAP expression is increased in the dorsal and ventral hippocampi of 5XFAD mice (as shown in FIG. 16E). And while treatment with TES slightly reduces GFAP expression in the dorsal hippocampus, this effect is not statistically significant.

Furthermore, TES improves spatial learning in female 5XFAD mice. Previous studies reported higher APP levels in the female than in male 5XFAD mice, likely due to the effects of an estrogen response element (ERE) present in the Thy1 promoter of the transgenes. The differential expression of APP transgene between male and female 5XFAD models may lead to gender differences in cognitive performance and plaque pathology. Hence, the effects of TES on memory improvements are also investigated in 6-month-old female 5XFAD mice.

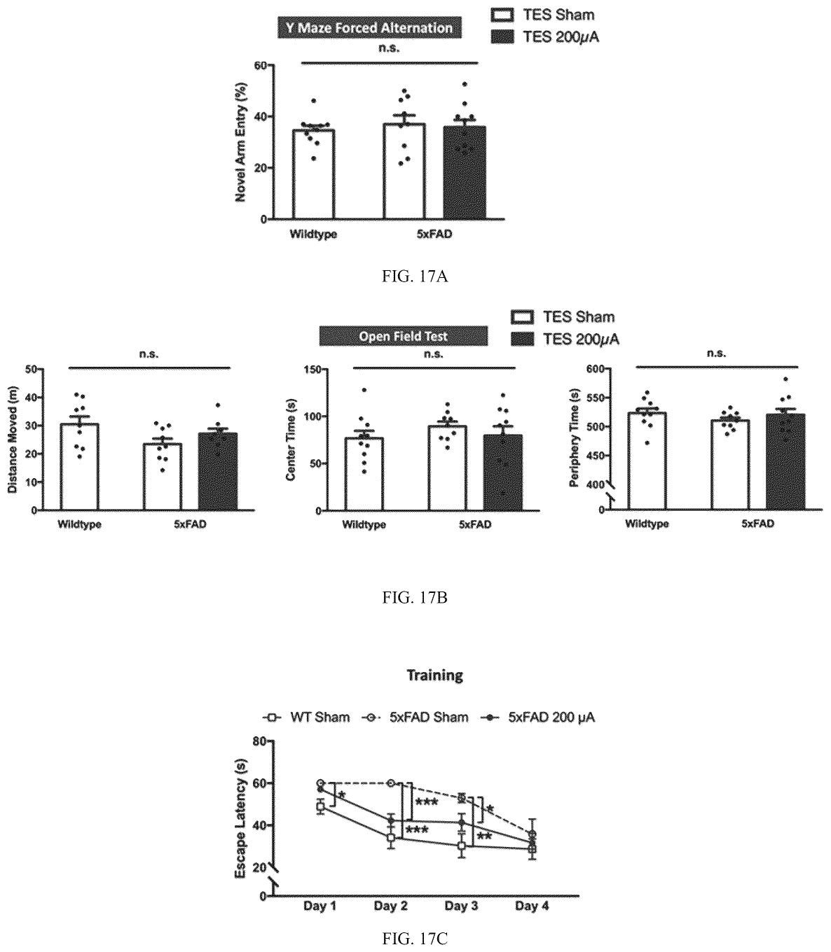

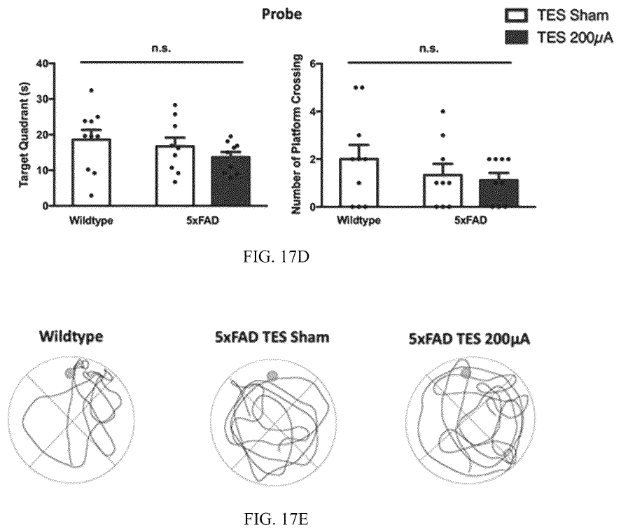

In the Y-maze forced alternation test, there are no significant differences in the percentage of novel arm entries between 5XFAD mice and their wild-type littermates, suggesting comparable spatial memory functions between the two groups (FIG. 17A). Stimulation at 200 μA does not alter behavior in the Y-maze test.

In the OFT, there are no significant differences in the total distance traveled and time spent in the center or periphery zones between 5XFAD mice and their wild-type littermates (FIG. 17B), indicating similar locomotion and anxiety levels. TES treatments in 5XFAD mice does not affect performance in the OFT.

During MWM training, spatial learning deficits are observed in female 5XFAD mice, as demonstrated by the longer time to locate the submerged platform compared with wild-type mice (FIG. 17C). The TES treatments in 5XFAD mice are able to effectively rescue learning ability, as indicated by a shorter escape latency. Nevertheless, no group differences are found in the time spent at the target quadrant or frequency of crossing the imaginary platform in the probe test (FIGS. 17D-17E), suggesting comparable spatial memory functions among wild-type, sham-, and TES-treated 5XFAD groups. Collectively, these results show that 6-month-old female 5XFAD mice have intact spatial memory, locomotor activity, and no emotional deficits; but show impaired spatial learning, which is rescued by TES treatment.

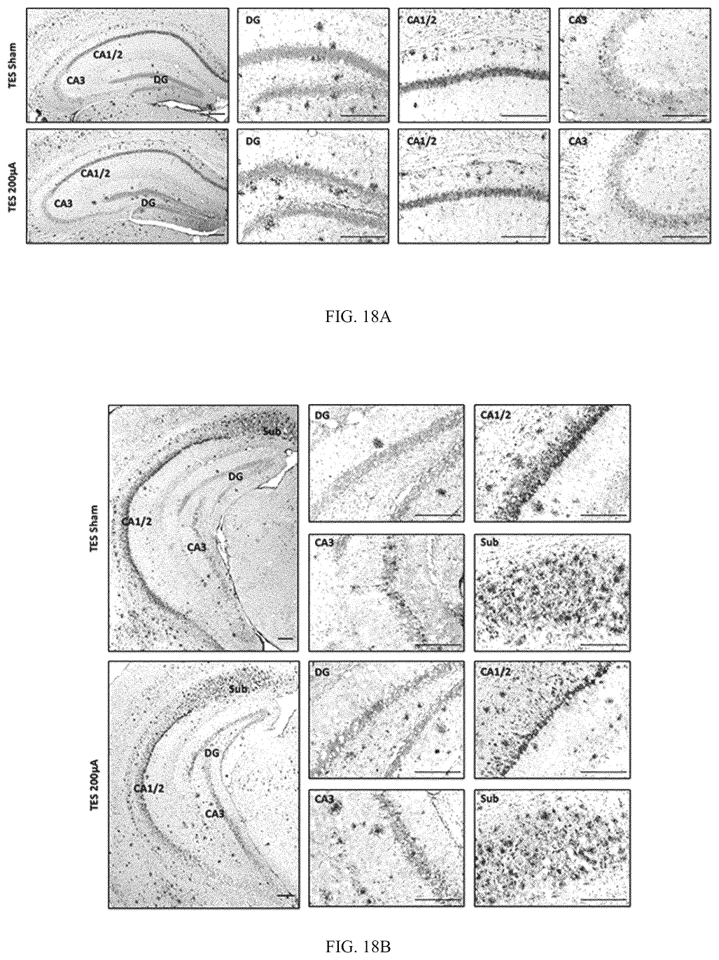

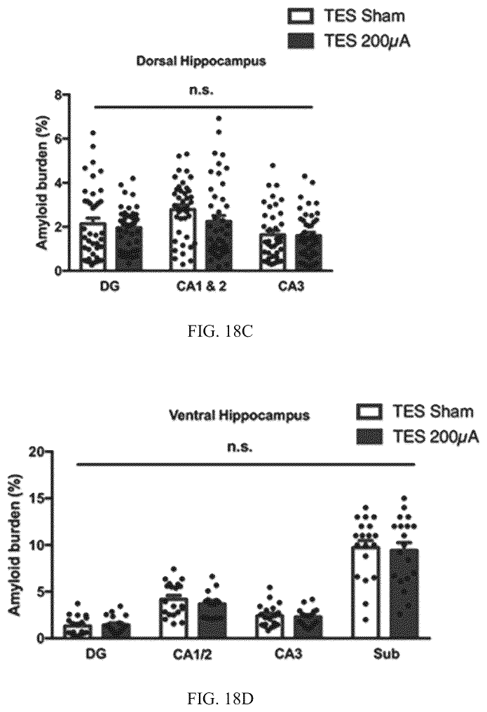

However, TES does not alter β-amyloid deposition in female 5XFAD mice. The accumulation of β-amyloid is observed in the DG and the CA1 & 2, CA3, and subiculum subregions in 6-month-old female 5XFAD mice (FIGS. 18A-18B). In contrast to the outcomes of male 5XFAD mice, TES administration does not significantly reduce amyloid burden in the hippocampus of female 5XFAD mice (FIGS. 18C-18D).

In summary, the present invention explores the effects of noninvasive electrical stimulation by TES on cognitive function in two animal models of cognitive decline, namely, aged mice and the 5XFAD model of AD. It is showed that TES improves memory functions in both animal models and reduce amyloid burden in the hippocampus of male 5XFAD mice. Moreover, there is an increase in the expression of PSD95, a postsynaptic marker, in the dorsal and ventral regions of the hippocampus in male 5XFAD mice.

The hippocampus is essential for learning and memory and is one of the first structures to undergo atrophy with aging. It is unsurprising that hippocampal-dependent spatial disorientation is among the most observable early manifestations of dementia. Therefore, spatial learning and memory in aged mice are assessed in the Y-maze and MWM tests to examine hippocampal-dependent learning and memory functions. It is confirmed that the aged mouse model exhibits impaired memory function in both behavioral tasks, indicating the use of aged mice as a valid animal model of dementia. Furthermore, it is observed that the aged mice travel a shorter distance in the open field and tend to spend less time in the center zone than young mice, which agrees with previous findings of decreased locomotor activity and increased anxiety-like behavior in older mice.

Enhanced performances in the MWM probe test in aged mice stimulated are found at either 100 or 200 μA, indicating that both amplitudes improve spatial memory retrieval in the aged model. Nevertheless, only TES at 200 μA reduces escape latency in the MWM learning phase, suggesting that stimulation at 200 μA is more effective in improving memory acquisition in older mice. Furthermore, the memory-enhancing effects of TES are not accompanied by altered locomotor activity or anxiety-like behavior in the OFT, implying that chronic TES at 100 or 200 μA does not result in adverse behavioral effects. It contradicts the corneal kindling studies that reported repeated subconvulsive TES caused anxiogenic-like responses and cognitive impairment in rodents. The adverse effects observed in the kindled model are likely triggered by the higher stimulation intensity ranging from 6 to 19 mA, which can cause tissue damage. This highlights a double-edged sword effect of TES, as it can generate different outcomes depending on the stimulation strength. Therefore, optimal stimulation parameters need to be established for efficacy.

Although aging is the major risk factor for dementia, one concern with using a natural aging mouse model is that the animals might not develop AD-like pathologies or cognitive dysfunction. Since AD is the major cause of dementia, the memory-enhancing effect of TES is further examined in a 5XFAD mouse model with early onset of AD. The 5XFAD model is an APP/PS1 double transgenic line harboring five familial AD mutations co-expressed to drive Aβ42 generation rapidly. 5XFAD mice develop age-dependent plaque deposition in the brain at 2 months and cognitive deficits from 4 to 6 months of age. In 6-month-old male 5XFAD mice given TES sham treatment, impaired spatial learning and memory, and a high plaque burden in the hippocampus are observed. It is found that TES at 200 μA is effective in normalizing memory function only in the Y-maze task (i.e., not in the MWM test). A possible explanation is that the swimming component in the MWM test induces anxiogenic and stress responses in mice, which in turn compromises their performance. As indicated by the similar behavioral responses across groups in the OFT, TES treatment does not affect locomotion or anxiety level in AD mice, which otherwise have interfered with the assessment of memory functions. To better understand the effect of TES on hippocampal-dependent memory in 5XFAD mice, the Barnes maze task is a less anxiogenic alternative to MWM that can be considered. Nevertheless, the present invention demonstrated that TES in male 5XFAD mice improves spatial memory retrieval in the Y-maze, which is accompanied by a reduction in amyloid plaque burden in various subregions of the dorsal and ventral hippocampi.

It is previously reported that memory performance in 5XFAD mice is negatively correlated with amyloid plaque burden. The present invention demonstrates the decreased 4g8 immunoreactivity after TES, suggesting that TES may be effective in reducing amyloid plaques and associated neurotoxic pathologies. This can serve as an underlying mechanism for the memory improvement induced by TES.

Besides advanced age, female gender is another major risk factor for AD. Therefore, the effects of TES in female 5XFAD mice is further examined. The behavioral observations in female 5XFAD mice are partially in line with those of male 5XFAD mice, with the sham-treated female transgenic mice present deficits in memory acquisition but not retrieval. Treatment with TES at 200 μA effectively restores MWM learning ability in 5XFAD females, although the effect on memory retrieval remains unclear. Furthermore, in agreement with the results in the aged model and male 5XFAD mice, TES in female 5XFAD mice does not lead to locomotion and anxiety changes in the OFT, which confirms the safety of TES at 200 μA. Although female 5XFAD mice does not exhibit pronounced memory impairment compared to their wild-type littermates, abundant plaque deposition is found in the dorsal and ventral hippocampi. Contrary to male 5XFAD mice, stimulation in female mice does not significantly reduce amyloid burden, which may imply that the improved learning observed in the MWM test might involve nonamyloidogenic mechanisms. Therefore, it is observed that there are gender differences in 5XFAD mice in response to TES treatment. This might be due to differences in body composition, variations in levels of endogenous sex hormones, influences from an ERE located in the Thy1 promoter of 5XFAD transgene, and/or individual variability resulting from the relatively small sample size in the embodiments (n=8-10). Nevertheless, gender-related differences in the efficacy and safety of TES treatment should be carefully considered for optimal stimulation parameters.

Along with plaque deposition, synaptic impairment and neuroinflammation are other hallmarks of AD, which are strongly associated with the severity of dementia. The postsynaptic degeneration is identified in the hippocampus of male 5XFAD mice in the embodiments, as demonstrated by a significant loss of PSD95 expression. PSD95 is a predominant scaffold protein of the postsynaptic density, which has functional roles in modulating synaptic plasticity and, therefore, memory formation. It is shown in the present invention that TES effectively enhances PSD95 expression in male 5XFAD mice, suggesting that TES improves cognitive function by restoring postsynaptic plasticity. Neuroinflammation in the brain appears to exert dual functions. While the activation of microglia and astrocytes has been found to confer neuroprotective effects by facilitating phagocytic clearance of β-amyloid, exacerbated glial-mediated neuroinflammation can drive synaptic loss, which accelerates AD progression. It is shown in the present invention that an increase in reactive astrocytes is determined by an increase in GFAP level in the hippocampus of 5XFAD mice. However, TES fails to elicit a significant reduction in GFAP expression. Furthermore, there are no changes observed in microglial IBA-1 expression in 5XFAD mice given sham or TES treatment. The results suggest that the clearance of plaque deposition and enhancement of PSD95 protein by TES could be mediated through non-anti-inflammatory mechanisms.

The present invention is the first investigating the effect of TES on cognitive functions in animal models and showing memory impairment.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

1. A transcorneal electrical stimulation (TES) apparatus for treating a brain disease, comprising:
an eye contacting interface comprising at least one active electrode;
an inactive reference electrode; and
a current source;
wherein the at least one active electrode and the inactive reference electrode are connected to the current source.

2. The apparatus of claim 1, further comprising a control unit configured to receive data related to the patient's brain activity and adjust the current parameters in real-time based on the received data.

3. The apparatus of claim 2, wherein the control unit is connected to the current source.

4. The apparatus of claim 1, wherein the current provider is programmable, allowing healthcare providers to customize treatment protocols for individual patients.

5. The apparatus of claim 1, wherein the brain disease comprises a depressive disorder and a dementia.

6. The apparatus of claim 1, wherein the current provider is an electrical pulse generator for controlling the delivery of electric current to the eye.

7. The apparatus of claim 1, wherein the eye contacting interface comprises a contact lens.

8. The apparatus of claim 7, wherein the contact lens is configured with a material that enhances electrode-skin contact and patient comfort.

9. A method of treating a brain disease in a patient in need utilizing the TES apparatus of claim 1, comprising:
placing the eye contacting interface upon a patient's eye and the inactive reference electrode on a skin area of the patient; and
providing a current through the electrodes for a duration.

10. The method of claim 9, wherein the current is a 100-200 μA current.

11. The method of claim 9, wherein the brain disease comprises a depressive disorder and a dementia.

12. The method of claim 9, further comprising adjusting the duration and intensity of the current based on the patient's need and capacity.

13. The method of claim 9, further comprising monitoring the patient's physiological responses during the treatment and adjusting the current accordingly.
